(19) United States
(12) Patent Application Publication
(10) Pub. No.: US 2007/0026398 A1
(43) Pub. Date: Feb. 1, 2007

(54) Protein tyrosine phosphatase-prl-1 a a marker and therapeutic target for pancreatic cancer

(76) Inventors: Amanda L Farnsworth, Northglenn, CO (US); Haiyong Han, Chandler, AZ (US); Hariprasad Vankayalapati, Salt Lake City, UT (US); Steven L. Warner, Tucson, AZ (US); Daniel D. Von Hoff, Scottsdale, AZ (US); David Bearss, Highland, UT (US)

Correspondence Address: FULBRIGHT & JAWORSKI L.L.P., 600 CONGRESS AVE., SUITE 2400, AUSTIN, TX, 78701, US

(21) Appl. No.: 10/547,488

(22) PCT Filed: Mar. 3, 2004
(86) PCT No.: PCT/US04/06269

§ 371 Date: Oct. 4, 2006

Related U.S. Application Data
(60) Provisional application No. 60/451,488, filed on Mar. 3, 2003.
(60) Provisional application No. 60/453,380, filed on Mar. 10, 2003.
(60) Provisional application No. 60/486,231, filed on Jul. 10, 2003.

(51) Int. Cl. C12Q 1/68; G01N 33/574

(52) U.S. Cl. 435/6; 435/7.23

(57) ABSTRACT

Using gene expression profiling, the present Invention identifies Protein tyrosine phosphatase IVA member 1 (PRL-1) as a diagnostic marker and therapeutic target for pancreatic cancer. The Invention therefore provides methods for prediction and detection of PRL-1 associated cancers, and evaluation of inhibitors of PRL-1. The Invention also provides a method of treating or preventing pancreatic cancer in a subject.

The present application claims the benefit of co-pending U.S. Provisional Patent Application Ser. No. 60/486,231, filed Jul. 10, 2003, U.S. Provisional Patent Application Ser. No. 60/453,380, filed Mar. 10, 2003, and U.S. Provisional Patent Application Ser. No. 60/451,488, filed Mar. 3, 2003, the entire disclosures of which are specifically incorporated herein by reference.

### BACKGROUND OF THE INVENTION

1. Field of the Invention

The present invention relates generally to the fields of molecular biology and cancer therapy. More particularly, it concerns diagnostic markers and drug targets for pancreatic cancer.

2. Description of Related Art

Pancreatic cancer is the fourth leading cause of cancer death among adults in the United States. In the year 2000 alone, an estimated 28,300 new cases of pancreatic cancer were diagnosed in the United States and nearly 28,200 patients were estimated to have died. Close to 90% of patients diagnosed with pancreatic cancer die within the first year following diagnosis. The deadliness of this disease has encouraged a search for factors that influence incidence and the molecular events that are involved in pancreatic tumor progression. At the molecular level, it is thought that the accumulation of defects in specific genes that contribute to the growth and development of normal tissue are responsible for the progression of cancer. Therefore, understanding the effects of genetic lesions that are common in the development of pancreas cancer will no doubt lead to new and more effective ways to diagnose, treat, and prevent this devastating disease.

The advent of cDNA microarray technology has made possible the identification and validation of new potential targets for drug development and analysis of the secondary effects of agents by monitoring changes in the expression of downstream genes. cDNA expression microarray analysis allows for the rapid identification of potential targets for drug development by examining the expression of thousands of genes in cancer cells versus normal cells. The changes in gene expression patterns from normal to tumor cells provide a background to determine what pathways are altered in cancer cells on a comprehensive scale.

Although some genes have been identified that are involved in pancreatic cancer, these discoveries have not proved beneficial in advancing the treatment and prevention of this disease. Thus, there still exists a need for additional disease markers and therapeutic targets in the field of pancreatic cancer.

### SUMMARY OF THE INVENTION

The present invention addresses the deficiencies in the art of an efficacious therapy for treating pancreatic cancer by investigating the molecular basis of the disease. In comparing pancreatic cancer cells to that of normal pancreas, by expression profiling, protein tyrosine phosphatase IVA member 1 (PRL-1) was identified as a diagnostic marker and a therapeutic target in treating this disease. Thus, the present invention provides a method of diagnosing or predicting development of pancreatic cancer in a subject comprising (a) obtaining a pancreatic cell sample from the subject; and (b) assessing PRL-1 activity or expression in the cell, wherein increased activity or expression of PRL-1 in the cell, when compared to a normal cell of the same type, indicates that the subject has or is at risk of developing pancreatic cancer.

A pancreatic cell sample embodied in the present invention may be precancerous pancreatic cell sample, a metastatic pancreatic cell sample, or a malignant pancreatic cell sample. Malignant pancreatic cell samples may further comprise a ductal adenocarcinoma cell sample, an intraductal papillary neoplasm cell sample, a papillary cystic neoplasm cell sample, a mucinous cystadenocarcinoma cell sample, a mucinous cystadenoma cell sample, an acinar carcinoma cell sample, an unclassified large cell carcinoma sample, a small cell carcinoma sample, or a pancreatoblastoma cell sample.

In other embodiments, the cell is a pancreatic tumor cell.

In a particular embodiment, the present invention comprises assessing PRL-1 expression or activity in a cell or sample, such as a tissue sample, by Northern blotting, quantitative RT-PCR, Western blotting or quantitative immunohistochemistry.

In some embodiments, the subject has previously been diagnosed with cancer or the subject has not previously been diagnosed with cancer and appears cancer free at the time of testing. In another embodiment, the present invention comprises administering a prophylactic cancer treatment, or a cancer therapy to the subject following testing. In other embodiments, the cancer therapy may be a chemotherapy, a radiotherapy, an immunotherapy, a gene therapy, a hormonal therapy or surgery.

In still another embodiment, the present invention provides a method of predicting the efficacy of a pancreatic cancer therapy comprising (a) administering a cancer therapy to the subject; (b) obtaining a pancreatic tumor cell sample from the subject; and (c) assessing PRL-1 activity or expression in the tumor cell of the sample, wherein decreased activity or expression of PRL-1 in the tumor cell, when compared to a tumor cell of the same type prior to treatment, indicates that the therapy is efficacious.

In further embodiments the present invention comprises assessing PRL-1 expression comprising measuring PRL-1 protein levels, or measuring PRL-1 transcript levels. In other embodiments, the present invention further comprises assessing PRL-1 activity or expression at multiple time points.

In still yet another embodiment, the present invention comprises a method of screening a candidate compound for anti-cancer activity comprising (a) providing a pancreatic cancer cell; (b) contacting the cell with a candidate compound; and (c) assessing the effect of the candidate compound on PRL-1 expression or activity, wherein a decrease in the amount of PRL-1 expression or activity, as compared to the amount of PRL-1 expression or activity in a similar cell not treated with the candidate compound, indicates that the candidate compound has anti-cancer activity.

The candidate compound of the present invention may be a protein, a nucleic acid or an organo-pharmaceutical.

In some embodiments of the invention the tumor cell may be selected from the group consisting of a precancerous pancreatic cell, a metastatic pancreatic cell, or a malignant pancreatic cell. The malignant pancreatic cell may further comprise a ductal adenocarcinoma cell, an intraductal papillary neoplasm cell, a papillary cystic neoplasm cell, a mucinous cystadenocarcinoma cell, a mucinous cystadenoma cell, an acinar carcinoma cell, an unclassified large cell carcinoma, a small cell carcinoma, or a pancreatoblastoma cell.

In further embodiments, a method of treating cancer comprises administering to a subject in need thereof a composition that inhibits PRL-1 activity or expression.

In still further embodiments, the candidate compound may be a protein, a nucleic acid or an organo-pharmaceutical. In yet a further embodiment, the protein is an antibody that binds immunologically to PRL-1. In still yet a further embodiment, the nucleic acid may be a PRL-1 antisense nucleic acid, a PRL-1 RNAi nucleic acid, or an antibody encoding a single-chain antibody that binds immunologically to PRL-1.

In some embodiments, the invention further comprises administering a second cancer therapy such as a chemotherapy, a radiotherapy, an immunotherapy, a gene therapy, a hormonal therapy or surgery to the subject.

In further embodiments, the composition of the invention may be administered more than once.

In a further embodiment, the present invention provides a method of diagnosing or predicting development of pancreatic cancer in a subject comprising subjecting the subject to whole body scanning for PRL-1 activity or expression in a cell.

In still a further embodiment, the present invention provides a method of monitoring an anticancer therapy comprising assessing the expression or function of PRL-1 in a pancreatic cancer cell of a subject following or during provision of the anticancer therapy.

It is contemplated that any method or composition described herein can be implemented with respect to any other method or composition described herein.

The use of the word “a” or “an” when used in conjunction with the term “comprising” in the claims and/or the specification may mean “one,” but it is also consistent with the meaning of “one or more,” “at least one,” and “one or more than one.”

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1. Schematic of gene expression profiling using a microarray.

FIG. 2. A hybridization of gene expression from BXPC-3 pancreatic cancer cells versus a Hela cell reference using the 5,760 gene chip.

FIGS. 3A-3C. Overexpression of genes identified from the microarray analysis. FIG. 3A—RT-PCR of various genes identified from the microarray analysis. FIG. 3B—RT-PCR of pancreatic cell lines overexpressing PRL-1. FIG. 3C—RT-PCR of pancreatic tumor samples showing overexpression of PRL-1.

FIG. 4. Pancreatic cancer tissue array. To aid in the validation of potential new targets for drug development, a pancreatic cancer tissue array was constructed consisting of 50 pancreatic cancer spots and 20 normal pancreas spots.

FIGS. 5A-5D. FIG. 5A—Antisense inhibitor AS-Prl-1C reduced mRNA levels of PRL-1. FIG. 5B—Real Time PCR data verifies that antisense oligo C targets PRL-1. FIG. 5C—Treatment of pancreatic cancer cells (MiaPaCa-2) with AS-Prl-1C results in arrest of cell growth. FIG. 5D—Pancreatic cancer cells (MiaPaCa-2) treated with AS-Prl-1C show a dramatic increase in apoptosis.

FIG. 6. Identification of siRNA sequences that reduced PRL-1 expression.

FIGS. 7A-7C. FIG. 7A—Western blot detection of His-tagged PRL-1 protein in TNT mixture. FIG. 7B—TNT product increased phosphatase activity. FIG. 7C—Inhibitory activity of tyrosine phosphatase inhibitors.

FIG. 8. Anti-PRL-1 activity of inhibitors.

FIG. 9. Clustal W alignment shows sequence identity and similarity between PRL-1 and the human phosphatases SHP2 and PTEN. The sequence alignment shows high homology in the active site of the phosphatase domains and increased variation outside of the active sites.

FIG. 10. 3D model of PRL-1 based on PTEN. The homology model of PRL-1 was constructed based on the above structure alignment using the modeling software in INSIGHT II. The PRL-1 homology model indicated a highly conserved hydrophobic core, but a changed specificity pocket without any major distortion of the geometry of the active site.

FIG. 11. Docking models of PRL-1 compounds.

FIG. 12. Lipid phosphatase activity of PRL-1.

FIGS. 13A-13C. Inhibition of cell proliferation by PRL-1 inhibitors using a SRB staining assay. FIG.13A—Inhibition of MiaPaCa-2 cell growth by UA668394. Cells were exposed to different doses of UA668394 (0.2 μM to 200 μM) for four days by SRB (Sulforhodamine B) staining. The estimated IC₅₀ is 1.2 μM. FIG. 13B—Inhibition of cell proliferation in pancreatic cancer cells Panc-1 and Mia PaCa-2 by the compound UA66839-1 analog. FIG. 13C—Inhibition of cell proliferation in pancreatic cancer cells Panc-l and Mia PaCa-2 by the compound UA668394-2 analog.

FIG. 14. Inhibition of PRL-1 expression by SMARTPool siRNA. MiaPaCa-2 cells were transiently transfected with either 50 nM (Lanes 1 and 6), 100 nM (Lanes 2 and 7) or 200 nM (Lanes 3 and 8) of the PRL-1 siRNA oligo mixture and harvested at either 48 hours (Lanes 1, 2 and 3) or 72 hours (Lanes 6, 7 and 8) after transfection. Lanes 4 and 9 control for 48 hour and 72 hour treatments, respectively. Lanes 5 and 10 are no treatment control (no siRNA and no Lipofectin) for 48 hour and 72 hour time points, respectively.

FIG. 15. PTEN Assay

FIG. 16. Mia PaCa-2 cells treated with the UA668394 compound was found to have an IC₅₀ of 1.2 μM. Cells treated with the UA19999 and UA45336 compounds showed an IC₅₀ of 120 μM and 95 μM respectively.

### DESCRIPTION OF ILLUSTRATIVE EMBODIMENTS

A. The Present Invention

As discussed above, one of the most deadly cancers is pancreatic cancers, with few patients living more than one year past initial diagnosis. Despite considerable focus on this disease, the prognosis for patients remains poor. Thus, intense research must be focused on cancers of the pancreas.

One aspect of this research is the search for a molecular basis for pancreatic cancer. The present inventors sought to examine the expression profiles of pancreatic cancer cells and compared these to normal cells. In so doing, they identified a group of dysregulated genes, the expression of which is greater or less in cancer cells than in a corresponding non-cancerous cell.

One of these genes, PRL-1, was highly overexpressed in most pancreatic cancer cells examined. Overexpression of the PRL-1 gene in pancreatic cancer cell lines was confirmed using Northern blotting and RT-PCR. To further ascertain that PRL-1 is a viable molecular target for cancer therapy, antisense oligonucleotides were used to inhibit the expression of PRL-1 in pancreatic cancer cells. Treated cells showed a significant increase in apoptosis and a decrease in accumulation of cells in the S phase. From these results, PRL-1 was confirmed as a diagnostic marker for pancreatic cancer, and a therapeutic target in treating this disease.

Thus, the present invention provides methods of assessing the activity or expression of PRL-1 protein or transcripts levels using a variety of techniques, the goal being the identification of cancers of pancreatic origin. The present invention also provides methods of screening for candidate inhibitors of PRL-1. Finally, the present invention provides methods of treating a cancer, in particular pancreatic cancer, by providing compositions that inhibit PRL-1 activity or expression, either as a single agent or in combination with other therapeutic agents. The details of the invention will be provided in the following materials.

B. Protein Tyrosine Phosphorylation and PRL-1 Phosphorylation of cellular proteins, particularly tyrosine phosphorylation, plays a central role in the regulation of a number of cellular processes, including cellular proliferation and differentiation (Tonks, 1993; Pawson et al., 1994). The protein tyrosine phosphatases (PTPase) belong to the protein phosphatase gene family. This phosphatase family consists of phosphatases that remove phosphate groups from protein tyrosine residues with high selectivity. One phosphorylated tyrosine residue may serve as a substrate, but another phosphotyrosine residue of the same protein may not. These phosphatases exist in a wide range of sizes and structural forms including transmembrane receptor-like and non-transmembrane forms. However, they all share homology within a region of 240 residues which defines a catalytic domain and contains a (I/V)HCXAGXXR(S/T)G consensus amino acid sequence near the C-terminus. Mutation of the active site cysteine residue abolishes this activity.

One member of this family of protein phosphatases is protein tyrosine phosphatase IVA member 1 (PRL-1), a non-transmembrane protein phosphatase. PRL-1 is a unique nuclear tyrosine phosphatase that controls cell growth. PRL-1 is 20 kDa in size, and is distinct from other protein tyrosine phosphatases of this family. PRL-1 has little homology to other PTPases outside the active site. However, PRL-1 is closely related to two other protein tyrosine phosphatases, PRL-2 and PRL-3. These PRL phosphatases contain a consensus motif for protein prenylation at the C-terminus (Zeng et al., 1998).

PRL-1 was initially identified as an immediate early gene involved in regenerating the liver (Diamond et al., 1996). This gene was also found to be expressed in mitogen-stimulated fibroblast. Stably transfected cells which overexpress PRL-1 demonstrate altered cellular growth and moiphology and a transformed phenotype. The expression of PRL-1 is associated with cell proliferation and differentation due to its ability to regulate the protein tyrosine phosphorylation and dephosphorylation of substrates that remain unknown. Overexpression of PRL-1 in epithelial cells has been shown to result in tumor formation in nude mice (Cates et al., 1996). It has also been suggested that PRL-1 function is regulated in a cell cycle dependent manner. PRL-1 has also been implicated in regulating progression through mitosis, possibly by modulating spindle dynamics (Wang et al., 2002). PRL-1 has been shown to be expressed in a number of tumor cell lines (Wang et al., 2002). Thus, the art suggests that PRL-1 has diverse roles in various tissues. At a minimum, it appears that PRL-1 is important in normal cellular growth control and may contribute to the tumorigenicity of some cancer cells (Diamond et al, 1994). The emergence of phosphatases, specifically protein tyrosine phosphatases, as potential therapeutic targets arose from recent studies with targeting PTP1B. Knockout, antisense and drug development studies have shown that down-regulation of PTP1B may be a good approach for treating diabetes and obesity (Elcheby et al., 1999). In cancer, several PTPs (e.g., PTP-a, PTP-E, Sapl, GLEPPI, PTP1 B) have been postulated to dephosphorylate and activate proto-oncogene Src-family kinases. PRL-3 and Cdc25B are other PTPs that have been shown to be specifically up-regulated in various tumor types.

C. Prognostic and Diagnostic Methods

A variety of methods known to those of ordinary skill in the art are available for assessing the activity or expression of a gene product in a cell, tissue sample or organism. The present invention embodies diagnostic methods and methods for assessing PRL-1 activity or expression comprising measuring PRL-1 protein or transcript levels. Methods of assessing for PRL-1 enzyme activity, or protein expression levels may also be employed. These methods are provided to identify subjects who both may be at risk for developing cancer, and who already have pancreatic cancer. In addition, these same methods may be applied to assess the efficacy of a cancer therapy.

Assays to assess the level of expression of a polypeptide are also well known to those of skill in the art. This can be accomplished also by assaying for PRL-1 mRNA levels, mRNA stability or turnover, as well as protein expression levels. It is further contemplated that any post-translational processing of PRL-1 may also be assessed, as well as whether it is being localized or regulated properly. In some cases an antibody that specifically binds PRL-1 may be used. Assays for PRL-1 activity also may be used.

1. Northern Blotting Techniques The present invention employs Northern blotting in assessing the expression of PRL-1 in a cancer or tumor cell. The techniques involved in Northern blotting are commonly used in molecular biology and are well known to one of skilled in the art. These techniques can be found in many standard books on molecular protocols (e.g., Sambrook et al., 2001). This technique allows for the detection of RNA i.e., hybridization with a labeled probe.

Briefly, RNA is separated by gel electrophoresis. The gel is then contacted with a membrane, such as nitrocellulose, permitting transfer of the nucleic acid and non-covalent binding. Subsequently, the membrane is incubated with, e.g., a chromophore-conjugated probe that is capable of hybridizing with a target amplification product. Detection is by exposure of the membrane to x-ray film or ion-emitting detection devices.

U.S. Pat. No. 5,279,721, incorporated by reference herein, discloses an apparatus and method for the automated electrophoresis and transfer of nucleic acids. The apparatus permits electrophoresis and blotting without external manipulation of the gel and is ideally suited to carrying out methods according to the present invention.

2. Quantitative RT-PCR

The present invention also employs quantitative RT-PCR in assessing the expression or activity of PRL-1 in a cancer or tumor cell. Reverse transcription (RT) of RNA to cDNA followed by relative quantitative PCR™ (RT-PCR) can be used to determine the relative concentrations of specific mRNA species, such as a PRL-1 transcript, isolated from a cell. By determining that the concentration of a specific mRNA species varies, it is shown that the gene encoding the specific mRNA species is differentially expressed

In PCR™, the number of molecules of the amplified target DNA increase by a factor approaching two with every cycle of the reaction until some reagent becomes limiting. Thereafter, the rate of amplification becomes increasingly diminished until there is not an increase in the amplified target between cycles. If one plots a graph on which the cycle number is on the X axis and the log of the concentration of the amplified target DNA is on the Y axis, one observes that a curved line of characteristic shape is formed by connecting the plotted points. Beginning with the first cycle, the slope of the line is positive and constant. This is said to be the linear portion of the curve. After some reagent becomes limiting, the slope of the line begins to decrease and eventually becomes zero. At this point the concentration of the amplified target DNA becomes asymptotic to some fixed value. This is said to be the plateau portion of the curve

The concentration of the target DNA in the linear portion of the PCR™ is directly proportional to the starting concentration of the target before the PCR™ was begun. By determining the concentration of the PCR™ products of the target DNA in PCR™ reactions that have completed the same number of cycles and are in their linear ranges, it is possible to determine the relative concentrations of the specific target sequence in the original DNA mixture. If the DNA mixtures are cDNAs synthesized from RNAs isolated from different cells, the relative abundances of the specific mRNA from which the target sequence was derived can be determined for the respective tissues or cells. This direct proportionality between the concentration of the PCR™ products and the relative mRNA abundances is only true in the linear range portion of the PCR™ reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR for a collection of RNA populations is that the concentrations of the amplified PCR™ products must be sampled when the PCR™ reactions are in the linear portion of their curves.

The second condition that must be met for an RT-PCR study to successfully determine the relative abundances of a particular mRNA species is that relative concentrations of the amplifiable cDNAs must be normalized to some independent standard. The goal of an RT-PCR study is to determine the abundance of a particular mRNA species relative to the average abundance of all mRNA species in the sample. In such studies, mRNAs for β-actin, asparagine synthetase and lipocortin II may be used as external and internal standards to which the relative abundance of other mRNAs are compared.

Most protocols for competitive PCR™ utilize internal PCR™ internal standards that are approximately as abundant as the target. These strategies are effective if the products of the PCR™ amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

The discussion above describes the theoretical considerations for an RT-PCR assay for clinically derived materials. The problems inherent in clinical samples are that they are of variable quantity (making normalization problematic), and that they are of variable quality (necessitating the co-amplification of a reliable internal control, preferably of larger size than the target).

Both of the foregoing problems are overcome if the RT-PCR is performed as a relative quantitative RT-PCR with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

Other studies are available that use a more conventional relative quantitative RT-PCR with an external standard protocol. These assays sample the PCR™ products in the linear portion of their amplification curves. The number of PCR™ cycles that are optimal for sampling must be empirically determined for each target cDNA fragment. In addition, the reverse transcriptase products of each RNA population isolated from the various tissue samples must be carefully normalized for equal concentrations of amplifiable cDNAs. This is very important since this assay measures absolute mRNA abundance. Absolute mRNA abundance can be used as a measure of differential gene expression only in normalized samples. While empirical determination of the linear range of the amplification curve and normalization of cDNA preparations are tedious and time consuming processes, the resulting RT-PCR assays can be superior to those derived from the relative quantitative RT-PCR with an internal standard. One reason for this is that without the internal standard/competitor, all of the reagents can be converted into a single PCR™ product in the linear range of the amplification curve, increasing the sensitivity of the assay. Another reason is that with only one PCR™ product, display of the product on an electrophoretic gel or some other display method becomes less complex, has less background and is easier to interpret.

3. Immunohistochemistry

The present invention also employs quantitative immunohistochemistry in assessing the expression of PRL-1 in a cancer or tumor cell.

Briefly, frozen-sections may be prepared by rehydrating 50 ng of frozen “pulverized” tumor at room temperature in phosphate buffered saline (PBS) in small plastic capsules; pelleting the particles by centrifugation; resuspending them in a viscous embedding medium (OCT); inverting the capsule and pelleting again by centrifugation; snap-freezing in −70° C. isopentane; cutting the plastic capsule and removing the frozen cylinder of tissue; securing the tissue cylinder on a cryostat microtome chuck; and cutting 25-50 serial sections containing an average of about 500 remarkably intact tumor cells.

Permanent-sections may be prepared by a similar method involving rehydration of the 50 mg sample in a plastic microfuge tube; pelleting; resuspending in 10% formalin for 4 h fixation; washing/pelleting; resuspending in warm 2.5% agar; pelleting; cooling in ice water to harden the agar; removing the tissue/agar block from the tube; infiltrating and embedding the block in paraffin; and cutting up to 50 serial permanent sections.

4. Western Blotting

The present invention also employs the use of Western blotting (immunoblotting) analysis to assess PRL-1 activity or expression in a cell such as a pancreatic cancer cell. This technique is well known to those of skill in the art, see U.S. Pat. No. 4,452,901 incorporated herein by reference and Sambrook et al. (2001). In brief, this technique generally comprises separating proteins in a sample such as a cell or tissue sample by SDS-PAGE gel electrophoresis. In SDS-PAGE proteins are separated on the basis of molecular weight, then are transferring to a suitable solid support, (such as a nitrocellulose filter, a nylon filter, or derivatized nylon filter), followed by incubation of the proteins on the solid support with antibodies that specifically bind to the proteins. For example, in the present invention, anti-PRL-1 antibodies specifically bind to PRL-1 proteins on the solid support. These antibodies may be directly labeled or alternatively may be subsequently detected using labeled antibodies (e.g. labeled sheep, goat, or mouse antibodies) that specifically bind to the anti-PRL-1.

5. ELISA

The present invention may also employ the use of immunoassays such as an enzyme linked immunosorbent assay (ELISA) in assessing the activity or expression of PRL-1 in a cancer or tumor cell. An ELISA generally involves the steps of coating, incubating and binding, washing to remove species that are non-specifically bound, and detecting the bound immune complexes. This technique is well known in the art, for example see U.S. Pat. No. 4,367,110 and Harlow and Lane, 1988.

In an ELISA assay, a PRL-1 protein sample may be immobilized onto a selected surface, preferably a surface exhibiting a protein affinity such as the wells of a polystyrene microtiter plate. After washing to remove incompletely adsorbed material, it is desirable to bind or coat the assay plate wells with a nonspecific protein that is known to be antigenically neutral with regard to the test antisera such as bovine serum albumin (BSA), casein or solutions of milk powder. This allows for blocking of nonspecific adsorption sites on the immobilizing surface and thus reduces the background caused by nonspecific binding of antisera onto the surface.

After binding of the antigenic material to the well, coating with a non-reactive material to reduce background, and washing to remove unbound material, the immobilizing surface is contacted with the antisera or clinical or biological extract to be tested in a manner conducive to immune complex (antigen/antibody) formation. Such conditions preferably include diluting the antisera with diluents such as BSA, bovine gamma globulin (BGG) and phosphate buffered saline (PBS)/Tween. These added agents also tend to assist in the reduction of nonspecific background. The layered antisera is then allowed to incubate for from 2 to 4 or more hours to allow effective binding, at temperatures preferably on the order of 25° C. to 37° C. (or overnight at 4° C.). Following incubation, the antisera-contacted surface is washed so as to remove non-immunocomplexed material. A preferred washing procedure includes washing with a solution such as PBS/Tween, or borate buffer.

Following formation of specific immunocomplexes between the test sample and the bound antigen and subsequent washing the occurrence and even amount of immunocomplex formation may be determined by subjecting the sample to a second antibody having specificity for the first. To provide a detecting means, the second antibody preferably has an associated enzyme that generates a color development upon incubating with an appropriate chromogenic substrate. Thus, for example, one will desire to contact and incubate the antisera-bound surface with a urease or peroxidase-conjugated anti-human IgG for a period of time and under conditions which favor the development of immunocomplex formation (e.g., incubation for 2 hours at room temperature in a PBS-containing solution such as PBS-Tween).

After incubation with the second enzyme-tagged antibody, and subsequent to washing to remove unbound material, the amount of label is quantified by incubation with a chromogenic substrate such as urea and bromocresol purple or 2,2′-azino-di-(3-ethyl-benzthiazoline-6-sulfonic acid (ABTS) and H₂O₂, in the case of peroxidase as the enzyme label. Quantification is then achieved by measuring the degree of color generation, e.g., using a visible spectra spectrophotometer. The use of labels for immunoassays are described in U.S. Pat. Nos. 5,310,687, 5,238,808 and 5,221,605.

Other immunodetection methods that may be contemplated in the present invention include radioimmunoassay (RIA), immunoradiometric assay, fluoroimmunoassay, chemiluminescent assay, bioluminescent assay. These methods are well known to those of ordinary skill and have been described in Doolittle et al. (1999); Gulbis et al. (1993); De Jager et al. (1993); and Nakamura et al (1987), each incorporated herein by reference.

6. Tissue Microarray Immunohistochemistry

Tissue microarray immunohistochemistry is a recently developed technique that enables the simultaneous examination of multiple tissues sections concurrently as compared to the more conventional technique of one section at a time. This technique is used for high throughput molecular profiling of tumor specimen (Kononen et al., 1998). More specifically, the present invention utilizes a pancreatic tumor tissue microarray containing different adenocarcinoma tissue samples, each of which having two representative 1.5 mm disks from the different areas of the same paraffin-embedded section. These pancreatic tissue microarrays may be used to verify the overexpression of other genes manifested in the cDNA microarray.

7. Determination of Circulating Cancer Cells

With the advent of enrichment techniques, detection of circulating cancer cells can be used for the early detection of cancer recurrence after treatment of a primary tumor, early diagnosis of metastasis and use in selection and monitoring of treatment strategies for various tumors (Martin et al., 1998; Wang et al., 2000; Hu et al., 2003). Anti-PRL antibodies of the present invention may be used in conjunction with cancer cell enrichment techniques in the detection of circulating pancreatic cancer cells. One suitable cell enrichment methodology is the magnetic-activated cell separation system as distributed by Miltenyi Biotec Inc. (Auburn, Calif.). This immunomagnetic method uses magnetically labeled anti-cytokeratin 8 antibodies to separate circulating cancer cells from other circulating cell types (see Martin et al., 1998; Hu et al., 2003). Pancreatic cancer cells express cytokeratin 8 (Rafie et al., 1992; Ditzel et al., 1997; Luttges et al., 1998). For example, blood samples (20-40 ml) are collected, treated with anticoagulant and stored for up to 23 hr. until further processing when they are spun down at 400 g for 35 minutes and the leukocyte-rich interphase cells are collected and permeabilized with PBS containing 0.5% BSA and 0.1% saponin and then fixed with 37% formaldehyde. After washing twice with PBS, 0.5% BSA, 0.5% saponin and 0.05% NaN₃, the cells are resuspended in 600 μl PBS, 0.5% BSA, 0.5% saponin and 0.05% NaN₃, and 200 μl FcR blocking reagent (Miltenyi Biotech) is added and the cancer cells directly magnetically labeled by the addition of 200 μl Cytokeratin Microbeads (Miltenyi Biotec, Auburn, Calif.) and incubating the cells for 45 min. at room temperature. The magnetically labeled cells are passed through a 30 μm filter and applied to a MACS MS enrichment column (Miltenyi Biotec), which is located within a magnetic field. Negative cells are washed of with PBS, 0.5% BSA, and 0.05% NaN₃, and then labeled cells are removed using the same buffer and the plunger supplied with the column after removal of the column from the magnetic field. Pancreatic cancer cells in this fraction can be detected by immunohistochemistry or flow cytometry using suitably labeled anti-PRL-1 antibodies. Alternatively, magnetically anti-PRL-1 antibodies may be used to enrich circulating pancreatic cancer cells.

An alternative enrichment technique is Circulating Cancer Cell Test (Cell Works Inc., Baltimore, Md.; see Wang et al., 2000). This procedure utilizes a double gradient sedimentation for the removal of most RBC and WBC as well as magnetic cell sorting for the additional removal of WBC before spreading the cancer cells onto a slide utilizing a cytospin apparatus. The fixed cells on the slide are then stained with a suitably anti-PRL-1 antibody and positive cells are automatically scanned with an spectroscopic microscope system, first in low magnification, where the fluorescent digital image is captured at a resolution of 0.2 μm using multiple excitation/emission wavelengths, then at higher resolution for further analysis. The system has automatic adjustment of exposure, focus and other parameters required for proper image acquisition and analysis to identify cancer cells and markers on the basis of intensity and blob analysis.

8. Whole Body Imaging

The present invention may further employ the use of whole body imaging techniques to identify subjects who have or may be at risk of developing cancer. Such diagnostic methods may employ positron emission tomography (PET) scanning, electron beam tomography (EBT) scanning, and MRI scanning. Essential to these methods is the use of labeled targeting agents, such as antibodies, that colocalize with PRL-1 in a quantitative fashion.

D. Screening Methods for PRL-1 Activity or Expression

1. Screening for Inhibitors of PRL-1

The present invention further comprises methods for identifying inhibitors of PRL-1 activity or expression. PRL-1 may be used as a target in screening for compounds that inhibit, decrease or down-regulate its expression or activity in cancer cells, such as pancreatic cancer cells. These assays may comprise random screening of large libraries of candidate substances. Alternatively, the assays may be used to focus on particular classes of compounds selected with an eye towards structural attributes that are believed to make them more likely to inhibit the function of PRL-1. By function, it is meant that one may assay for inhibition of expression of PRL-1 in cancer cells, increase apoptosis, or inhibition of the ability of the PRL-1 enzyme to cleave phosphatases off of the substrate.

To identify a PRL-1 inhibitor, one generally will determine PRL-1 activity or expression in the presence and absence of the candidate substance, wherein an inhibitor is defined as any substance that down-regulates, reduces, inhibits or decreases PRL-1 activity or expression. For example, a method may generally comprise:
a) providing a cell;
b) contacting the cell with a candidate compound; and
c) assessing the effect of the candidate compound on PRL-1 expression or activity,
   wherein a decrease in the amount of PRL-1 expression or activity, as compared to the amount of PRL-1 expression or activity in a similar cell not treated with the candidate compound, indicates that the candidate compound has anti-cancer activity.

Assays may be conducted in cell free systems, in isolated cells, or in organisms including transgenic animals. It will, of course, be understood that all the screening methods of the present invention are useful in themselves notwithstanding the fact that effective candidates may not be found. The invention provides methods for screening for such candidates, not solely methods of finding them.

a. Inhibitors

As used herein the term “candidate substance” or “candidate compound” refers to any molecule that may potentially inhibit the expression or activity of PRL-1. A PRL-1 inhibitor, may be a compound that overall affects an inhibition of PRL-1 activity, which may be accomplished by inhibiting PRL-1 expression, translocation or transport, function, expression, post-translational modification, location, or more directly by preventing its activity, such as by binding PRL-1. Any compound or molecule described in the methods and compositions herein may be an inhibitor of PRL-1 activity or expression.

The candidate substance may be a protein or fragment thereof, a small molecule, or even a nucleic acid molecule. It may prove to be the case that the most useful pharmacological compounds will be compounds that are structurally related to PRL-1 or other protein tyrosine phosphatases, or that binds PRL-1. Using lead compounds to help develop improved compounds is known as “rational drug design” and include not only comparisons with known inhibitors, but predictions relating to the structure of target molecules.

Candidate compounds or inhibitors of the present invention will likely function inhibit decrease or down-regulate the expression or activity of PRL-1 in a cancer cell such as a pancreatic cancer cell. Such candidate compounds may be inhibitors or regulators of protein tyrosine phosphatases; may have the ability to remove a phosphate from proteins or peptides containing phosphotyrosine; or may likely be involved in controlling cellular proliferation in a cancer or tumor cell, such as pancreatic cancer cells. These candidate compounds may be antisense molecules, ribozymes, interfering RNAs, antibodies (including single chain antibodies), or organopharmaceuticals, but are not limited to such.

b. Rational Drug Design

The present invention also provides methods for developing drugs that inhibit PRL-1 activity or expression that may be used to treat a cancer, such as pancreatic cancer. One such method involves the prediction of the three dimensional structure of a validated protein tyrosine phosphatase target using molecular modeling and computer stimulations. The resulting structure may then be used in docking studies to identify potential small molecule inhibitors that bind in the enzyme's active site with favorable binding energies. Inhibitors identified may then be tested in biochemical assays to further identify PRL-1 drug target for pancreatic cancer treatment.

Rational drug design is therefore used to produce structural analogs of phosphorylated substrates for PRL-1. By creating such analogs, it is possible to fashion drugs which are more active or stable than the natural molecules, which have different susceptibility to alteration or which may affect the function of various other molecules. In one approach, one would generate a three-dimensional structure for the PRL-1 targets of the invention or a fragment thereof. This could be accomplished by X-ray crystallography, computer modeling or by a combination of both approaches.

It also is possible to use antibodies to ascertain the structure of a target compound inhibitor. In principle, this approach yields a phannacore upon which subsequent drug design can be based. It is possible to bypass protein crystallography altogether by generating anti-idiotypic antibodies to a functional, pharmacologically active antibody. As a mirror image of a mirror image, the binding site of anti-idiotype would be expected to be an analog of the original antigen. The anti-idiotype could then be used to identify and isolate peptides from banks of chemically- or biologically-produced peptides. Selected peptides would then serve as the pharmacore. Anti-idiotypes may be generated using the methods described herein for producing antibodies, using an antibody as the antigen.

On the other hand, one may simply acquire, from various commercial sources, small molecule libraries that are believed to meet the basic criteria for useful drugs in an effort to “brute force” the identification of useful compounds. Screening of such libraries, including combinatorially generated libraries (e.g., peptide libraries), is a rapid and efficient way to screen large number of related (and unrelated) compounds for activity. Combinatorial approaches also lend themselves to rapid evolution of potential drugs by the creation of second, third and fourth generation compounds modeled of active, but otherwise undesirable compounds.

Candidate compounds may include fragments or parts of naturally-occurring compounds, or may be found as active combinations of known compounds, which are otherwise inactive. It is proposed that compounds isolated from natural sources, such as animals, bacteria, fungi, plant sources, including leaves and bark, and marine samples may be assayed as candidates for the presence of potentially useful pharmaceutical agents. It will be understood that the pharmaceutical agents to be screened could also be derived or synthesized from chemical compositions or man-made compounds. Thus, it is understood that the candidate substance identified by the present invention may be peptide, polypeptide, polynucleotide, small molecule inhibitors or any other compounds that may be designed through rational drug design starting from known inhibitors or stimulators.

Other suitable compounds include antisense molecules, ribozymes, and antibodies (including single chain antibodies), each of which would be specific for the target molecule. Such compounds are described in greater detail elsewhere in this document. For example, an antisense molecule that bound to a translational or transcriptional start site, or splice junctions, would be ideal candidate inhibitors.

In addition to the inhibiting compounds initially identified, the inventors also contemplate that other sterically similar compounds may be formulated to mimic the key portions of the structure of the inhibitors. Such compounds, which may include peptidomimetics of peptide inhibitors, may be used in the same manner as the initial inhibitors.

An inhibitor according to the present invention may be one which exerts its inhibitory or activating effect upstream, downstream or directly on PRL-1 or other related phosphates of this gene family. Regardless of the type of inhibitor identified by the present screening methods, the effect of the inhibition by such a compound results in the regulation in PRL-1 activity or expression as compared to that observed in the absence of the added candidate substance.

The term “drug” is intended to refer to a chemical entity, whether in the solid, liquid, or gaseous phase which is capable of providing a desired therapeutic effect when administered to a subject. The term “drug” should be read to include synthetic compounds, natural products and macromolecular entities such as polypeptides, polynucleotides, or lipids and also small entities such as neurotransmitters, ligands, hormones or elemental compounds. The term “drug” is meant to refer to that compound whether it is in a crude mixture or purified and isolated.

c. Bioisosterism

The present invention also contemplates the application of bioisosterism, the concept of isosterism to modify biological activity of a lead compound, in developing drugs that cacn inhibit PRL-1 activity or expression that may be used as therapeutic agents. As discussed above, a lead compound with a desired pharmacological activity may have associated with it undesirable side effects, characteristics that limit its bioavailability, or structural features which adversely influence its metabolism and excretion from the body. Bioisosterism represents one approach used in the art for the rational modification of lead compounds into safer and more clinically effective agents (Patani and LaVoie, 1996). The ability of a group of bioisosteres to elicit similar biological activity has been attributed to common physicochemical properties such as electro-negativity, steric size, and lipophilicity. Bioisosteric replacements of functional groups based on the understanding of the pharmacophore and the physicochemical properties of the bioisosteres have enhanced the potential for the successful development of new clinical agents. A critical component for bioisosterism is that bioisosteres affect the same pharmacological target as agonists or antagonists and, thereby, have biological properties which are related to each other.

Bioisosteres are classified as either classical or nonclassical. Classical bioisosteres have been traditionally divided into several distinct categories: (a) monovalent atoms or groups; (b) divalent atoms or groups; (c) trivalent atoms or groups; (d) tetrasubstituted atoms; and (e) ring equivalents. Nonclassical bioisosteres can be divided into (a) rings vs noncyclic structures; and (b) exchangeable groups. Nonclassical isosteres differ from that of the classical bioisosteres in that they do not obey the steric and electronic definition of classical isosteres. A notable characteristic of nonclassical bioisosteres is that they do not have the same number of atoms as the substituent or moiety for which they are used as a replacement.

In the present invention the application of bioisosterism has been employed in developing agents that can inhibit PRL-1 activity or expression. For example, the pharmacophore of the a lead compound, UA668394, may be exploited using the concept of bioisosterism to develop the analogs UA668394-1 and UA668394-2 as provided below: 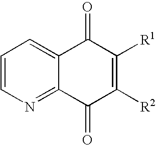
wherein R¹ is hydrogen, halogen, thiol, trifluoromethyl, or hydroxyl and R² is a hydrogen, halogen, thiol, hydroxyl or trifluoromethyl, 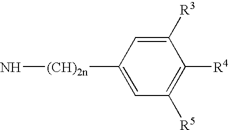
wherein R³ and R⁵ are independently halogen, thiol, hydroxy or trifluoromethyl, and R⁴ is hydroxyl, halogen, thiol, trifluoromethyl, CH₂OH, NHCONH₂, NHSO₂CH₃, or NHCN, 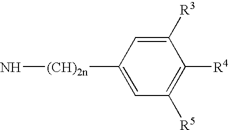
wherein R³ and R⁵ are independently halogen, thiol, hydroxyl or trifluoromethyl, and R⁴ is hydroxyl, halogen, thiol, trifluoromethyl, CH₂OH, NHCONH₂, NHSO₂CH₃, or NHCN.

2. Phosphatase Assays

a. Tyrosine Phosphatase Assay

Assays that measure the removal of phosphates from proteins or peptides containing phosphotyrosine may also be employed in the present invention. One method of screening for drug targets would involve measuring inhibition of PRL-1-mediated tyrosine dephosphorylation. This assay detects the amount of free phosphatase generated in a reaction by measuring the absorbance of a molybdate:malachite green:phosphate complex. This assay detects the activity of protein tyrosine phosphatases. Such assays or systems are commercially available from suppliers such as Promega (Madison, Wis.) or Applied Biosystems (Foster City, Calif.).

b. DiFMUP Assay

Another assay employed in the present invention is an improved method for measuring protein phosphatases for high-throughput screening involving 6,8-difluoro-4-methylumbelliferyl phosphate (DiFMUP). DiFMUP can assay both acid and alkaline phosphatase activity. The hydrolysis product of DiFMUP to DiF4MU exhibits both a lower pka (4.9 versus 7.8) and a higher fluorescence quantum yield (0.89 versus 0.63) than the hydrolysis product of MUP. The lower pka of its hydrolysis product makes DiFMUP a sensitive substrate for acid phosphatases, which is not possible with MUP because its fluorescence must be measured at alkaline pH. Furthermore, with its high quantum yield, DiFMUP increases the sensitivity of both acid and alkaline phosphatase measurements. As with fluorinated fluorescein derivatives (i.e., Oregon Green dyes) fluorination reduces the susceptibility of the methylumbelliferone fluorophore to photobleaching effects without significantly affecting the extinction coefficient or excitation/emission maxima. DiFMUP enables the quantitation of as little as 1.0 pg/ml alkaline phosphatase.

For example, in the present invention, a bacterial expression system may be employed (i.e., pProEx vector) from which recombinant His-tagged PRl-1 protein may be obtained and purified using a column (i.e., a nickel column). The PRl-1 enzymatic activity in the presence of a drug compound of interest and in combination with DiFMUP substrate may be incubated (about 1 h) and the dephosphorylated substrate detected.

3. In Vitro Assays

A quick, inexpensive and easy assay to run is an in vitro assay. Such assays generally use isolated molecules, and can be run quickly and in large numbers, thereby increasing the amount of information obtainable in a short period of time. A variety of vessels may be used to run the assays, including test tubes, plates, dishes and other surfaces such as dipsticks or beads.

One example of a cell-free assay is a binding assay. While not directly addressing function, the ability of a compound to bind to a target molecule such as PRL-1 in a specific fashion is strong evidence of a related biological effect, which can be assessed in follow on screens. For example, binding of a molecule to PRL-1 may, in and of itself, be inhibitory, due to steric, allosteric or charge-charge interactions. The PRL-1 may be either free in solution, fixed to a support, expressed in or on the surface of a cell. Either the PRL-1 or the compound may be labeled, thereby permitting measuring of the binding. Competitive binding formats can be performed in which one of the agents is labeled, and one may measure the amount of free label versus bound label to determine the effect on binding.

A technique for high throughput screening of compounds is described in WO 84/03564. Large numbers of small peptide test compounds are synthesized on a solid substrate, such as plastic pins or some other surface. Bound polypeptide is detected by various methods.

4. In Cyto Assays

The present invention also contemplates the screening of compounds for their ability to inhibit PRL-1 in cells. Various cell lines can be utilized for such screening assays, including cells specifically engineered for this purpose. The present invention particularly contemplates the use of pancreatic cancer cells, which express a higher level of PRL-1 activity, and thus may provide an easier baseline for measurement. Depending on the assay, culture may be required. The cell is examined using any of a number of different physiologic assays. Alternatively, molecular analysis may be performed, for example, looking at protein expression, mRNA expression (including differential display of whole cell or polyA RNA) and others by methods as described herein and that are well known to those of skill in the art.

5. In Vivo Assays

In vivo assays involve the use of various animal models, including transgenic animals that have been engineered to have specific defects such as PRL-1 overexpression, or that carry markers that can be used to measure the ability of a candidate substance to reach and effect different cells within the organism. Due to their size, ease of handling, and information on their physiology and genetic make-up, mice are a preferred embodiment, especially for transgenics. However, other animals are suitable as well, including rats, rabbits, hamsters, guinea pigs, gerbils, woodchucks, cats, dogs, sheep, goats, pigs, cows, horses and monkeys (including chimps, gibbons and baboons). Assays for inhibitors may be conducted using an animal model derived from any of these species.

In such assays, one or more candidate substances are administered to an animal, and the ability of the candidate substance(s) to alter one or more characteristics, as compared to a similar animal not treated with the candidate substance(s), identifies an inhibitor. The characteristics may be any of those discussed above with regard to PRL-1 expression or function, or it may be broader in the sense of “treating” the condition present in the animal.

Treatment of these animals with test compounds will involve the administration of the compound, in an appropriate form, to the animal. Administration will be by any route that could be utilized for clinical or non-clinical purposes, including but not limited to oral, nasal, buccal, or even topical. Alternatively, administration may be by intratracheal instillation, bronchial instillation, intradermal, subcutaneous, intramuscular, intraperitoneal or intravenous injection. Specifically contemplated routes are systemic intravenous injection, regional administration via blood or lymph supply, or directly to an affected site.

Determining the effectiveness of a compound in vivo may involve measuring toxicity and dose response can be performed in animals in a more meaningful fashion than in in vitro or in cyto assays.

E. Cancer Treatment

The present invention embodies a method of treating cancer such as pancreatic cancer, by the delivery of a PRL-1 inhibitor to a subject having a cancer. Examples of cancers contemplated for treatment include leukemia, ovarian cancer, breast cancer, lung cancer, colon cancer, liver cancer, prostate cancer, testicular cancer, stomach cancer, brain cancer, bladder cancer, head and neck cancer, melanoma, and any other cancer that may be treated by inhibiting or decreasing the activity of PRL-1 activity.

1. PRL-1 Inhibitors

a. Antisense

Antisense methodology takes advantage of the fact that nucleic acids tend to pair with “complementary” sequences. By complementary, it is meant that polynucleotides are those which are capable of base-pairing according to the standard Watson-Crick complementarity rules. That is, the larger purines will base pair with the smaller pyrimidines to form combinations of guanine paired with cytosine (G:C) and adenine paired with either thymine (A:T) in the case of DNA, or adenine paired with uracil (A:U) in the case of RNA. Inclusion of less common bases such as inosine, 5-methylcytosine, 6-methyladenine, hypoxanthine and others in hybridizing sequences does not interfere with pairing.

Targeting double-stranded (ds) DNA with polynucleotides leads to triple-helix formation; targeting RNA will lead to double-helix formation. Antisense polynucleotides, when introduced into a target cell, specifically bind to their target polynucleotide and interfere with transcription, RNA processing, transport, translation and/or stability. Antisense RNA constructs, or DNA encoding such antisense RNAs, may be employed to inhibit gene transcription or translation or both within a host cell, either in vitro or in vivo, such as within a host animal, including a human subject.

Antisense constructs may be designed to bind to the promoter and other control regions, exons, introns or even exon-intron boundaries of a gene. It is contemplated that the most effective antisense constructs may include regions complementary to intron/exon splice junctions. Thus, antisense constructs with complementarity to regions within 50-200 bases of an intron-exon splice junction may be used. It has been observed that some exon sequences can be included in the construct without seriously affecting the target selectivity thereof. The amount of exonic material included will vary depending on the particular exon and intron sequences used. One can readily test whether too much exon DNA is included simply by testing the constructs in vitro to determine whether normal cellular function is affected or whether the expression of related genes having complementary sequences is affected.

As stated above, “complementary” or “antisense” means polynucleotide sequences that are substantially complementary over their entire length and have very few base mismatches. For example, sequences of fifteen bases in length may be termed complementary when they have complementary nucleotides at thirteen or fourteen positions. Naturally, sequences which are completely complementary will be sequences which are entirely complementary throughout their entire length and have no base mismatches. Other sequences with lower degrees of homology also are contemplated. For example, an antisense construct which has limited regions of high homology, but also contains a non-homologous region (e.g., ribozyme) could be designed. These molecules, though having less than 50% homology, would bind to target sequences under appropriate conditions.

It may be advantageous to combine portions of genomic DNA with cDNA or synthetic sequences to generate specific constructs. For example, where an intron is desired in the ultimate construct, a genomic clone will need to be used. The cDNA or a synthesized polynucleotide may provide more convenient restriction sites for the remaining portion of the construct and, therefore, would be used for the rest of the sequence.

b. Ribozymes

The present invention also contemplates the use of PRL-1-specific ribozymes to down-regulate or inhibit PRL-1 expression. Ribozymes are RNA-protein complexes that cleave nucleic acids in a site-specific fashion. Ribozymes have specific catalytic domains that possess endonuclease activity (Kim and Cech, 1987; Forster and Symons, 1987). For example, a large number of ribozymes accelerate phosphoester transfer reactions with a high degree of specificity, often cleaving only one of several phosphoesters in an oligonucleotide substrate (Cech et. al., 1981; Michel and Westhof, 1990; Reinhold-Hurek and Shub, 1992). This specificity has been attributed to the requirement that the substrate bind via specific base-pairing interactions to the internal guide sequence (“IGS”) of the ribozyme prior to chemical reaction.

Ribozyme catalysis has primarily been observed as part of sequence specific cleavage/ligation reactions involving nucleic acids (Joyce, 1989; Cech et. al., 1981). For example, U.S. Pat. No. 5,354,855 reports that certain ribozymes can act as endonucleases with a sequence specificity greater than that of known ribonucleases and approaching that of the DNA restriction enzymes. Thus, sequence-specific ribozyme-mediated inhibition of gene expression (Scanlon et al., 1991; Sarver et. al., 1990; Sioud et. al., 1992) is particularly suited to therapeutic applications of the present invention. It has been reported that ribozymes elicited genetic changes in some cell lines to which they were applied; the altered genes included the oncogenes H-ras, c-fos and genes of HIV. Most of this work involved the modification of a target mRNA, based on a specific mutant codon that is cleaved by a specific ribozyme. In light of the information included herein and the knowledge of one of ordinary skill in the art, the preparation and use of additional ribozymes that are specifically targeted to a given gene will now be straightforward.

Several different ribozyme motifs have been described with RNA cleavage activity (reviewed in Symons, 1992). Examples that would be expected to function equivalently for the down-regulation or inhibition of PRl-1 include sequences from the Group I self splicing introns including tobacco ringspot virus (Prody et. al., 1986), avocado sunblotch viroid (Palukaitis et. al., 1979), and Lucerne transient streak virus (Forster and Symons, 1987). Sequences from these and related viruses are referred to as hammerhead ribozymes based on a predicted folded secondary structure.

Other suitable ribozymes include sequences from RNase P with RNA cleavage activity (Yuan et al., 1992; Yuan and Altman, 1994), hairpin ribozyme structures (Berzal-Herranz et al., 1992; Chowrira et al., 1993) and hepatitis virus based ribozymes (Perrotta and Been, 1992). The general design and optimization of ribozyme directed RNA cleavage activity has been discussed in detail (Haseloff and Gerlach, 1988; Symons, 1992; Chowrira, et al., 1994; and Thompson, et al., 1995).

The other variable on ribozyme design is the selection of a cleavage site on a given target RNA. Ribozymes are targeted to a given sequence by virtue of annealing to a site by complimentary base pair interactions. Two stretches of homology are required for this targeting. These stretches of homologous sequences flank the catalytic ribozyme structure defined above. Each stretch of homologous sequence can vary in length from 7 to 15 nucleotides. The only requirement for defining the homologous sequences is that, on the target RNA, they are separated by a specific sequence which is the cleavage site. For hammerhead ribozymes, the cleavage site is a dinucleotide sequence on the target RNA, uracil (U) followed by either an adenine, cytosine or uracil (A,C or U; Perriman, et al., 1992; Thompson, et al., 1995). The frequency of this dinucleotide occurring in any given RNA is statistically 3 out of 16. Therefore, for a given target mRNA of 1000 bases, 187 dinucleotide cleavage sites are statistically possible.

Designing and testing ribozymes for efficient cleavage of a target RNA is a process well known to those skilled in the art. Examples of scientific methods for designing and testing ribozymes are described by Chowrira et al. (1994) and Lieber and Strauss (1995), each incorporated by reference. The identification of operative and preferred sequences for use in PRL-1-targeted ribozymes is simply a matter of preparing and testing a given sequence, and is a routinely practiced “screening” method known to those of skill in the art.

C. RNA Interference (RNAi)

RNA interference (also referred to as “RNA-mediated interference” or RNAi) is a mechanism by which gene expression can be reduced or eliminated. Double stranded RNA (dsRNA) has been observed to mediate the reduction, which is a multi-step process. dsRNA activates post-transcriptional gene expression surveillance mechanisms that appear to function to defend cells from virus infection and transposon activity. (Fire et al., 1998; Grishok et al., 2000; Ketting et al., 1999; Lin et al., 1999; Montgomery et al., 1998; Sharp et al., 2000; Tabara et al., 1999). Activation of these mechanisms targets mature, dsRNA-complementary mRNA for destruction. RNAi offers major experimental advantages for study of gene function. These advantages include a very high specificity, ease of movement across cell membranes, and prolonged down-regulation of the targeted gene. (Fire et al., 1998; Grishok et al., 2000; Ketting et al., 1999; Lin et al., 1999; Montgomery et al., 1998; Sharp, 1999; Sharp et al., 2000; Tabara et al., 1999). Moreover, dsRNA has been shown to silence genes in a wide range of systems, including plants, protozoans, fungi, C. elegans, Trypanasoma, Drosophila, and mammals (Grishok et al., 2000; Sharp, 1999; Sharp et al., 2000; Elbashir et al., 2001). It is generally accepted that RNAi acts post-transcriptionally, targeting RNA transcripts for degradation. It appears that both nuclear and cytoplasmic RNA can be targeted. (Bosher et al., 2000).

siRNAs must be designed so that they are specific and effective in suppressing the expression of the genes of interest. Methods of selecting the target sequences, i.e. those sequences present in the gene or genes of interest to which the siRNAs will guide the degradative machinery, are directed to avoiding sequences that may interfere with the siRNA's guide function while including sequences that are specific to the gene or genes. Typically, siRNA target sequences of about 21 to 23 nucleotides in length are most effective. This length reflects the lengths of digestion products resulting from the processing of much longer RNAs as described above. (Montgomery et al., 1998).

The making of siRNAs has been mainly through direct chemical synthesis; through processing of longer, double stranded RNAs through exposure to Drosophila embryo lysates; or through an in vitro system derived from S2 cells. Use of cell lysates or in vitro processing may further involve the subsequent isolation of the short, 21-23 nucleotide siRNAs from the lysate, etc., making the process somewhat cumbersome and expensive. Chemical synthesis proceeds by making two single stranded RNA-oligomers followed by the annealing of the two single stranded oligomers into a double stranded RNA. Methods of chemical synthesis are diverse. Non-limiting examples are provided in U.S. Pat. Nos. 5,889,136; 4,415,732; 4,458,066, expressly incorporated herein by reference, and in Wincott et. al. (1995).

Several further modifications to siRNA sequences have been suggested in order to alter their stability or improve their effectiveness. It is suggested that synthetic complementary 21-mer RNAs having di-nucleotide overhangs (i.e., 19 complementary nucleotides +3′ non-complementaiy dimers) may provide the greatest level of suppression. These protocols primarily use a sequence of two (2′-deoxy) thymidine nucleotides as the di-nucleotide overhangs. These dinucleotide overhangs are often written as dTdT to distinguish them from the typical nucleotides incorporated into RNA. The literature has indicated that the use of dT overhangs is primarily motivated by the need to reduce the cost of the chemically synthesized RNAs. It is also suggested that the dTdT overhangs might be more stable than UU overhangs, though the data available shows only a slight (<20%) improvement of the dTdT overhang compared to an siRNA with a UU overhang.

Chemically synthesized siRNAs are found to work optimally when they are in cell culture at concentrations of 25-100 nM. This had been demonstrated by Elbashir et. al. wherein concentrations of about 100 nM achieved effective suppression of expression in mammalian cells. siRNAs have been most effective in mammalian cell culture at about 100 nM. In several instances, however, lower concentrations of chemically synthesized siRNA have been used (Caplen et. al., 2000; Elbashir et. al., 2001).

WO 99/32619 and WO 01/68836 suggest that RNA for use in siRNA may be chemically or enzymatically synthesized. Both of these texts are incorporated herein in their entirety by reference. The enzymatic synthesis contemplated in these references is by a cellular RNA polymerase or a bacteriophage RNA polymerase (e.g. T3, T7, SP6) via the use and production of an expression construct as is known in the art. For example, see U.S. Pat. No. 5,795,715. The contemplated constructs provide templates that produce RNAs that contain nucleotide sequences identical to a portion of the target gene. The length of identical sequences provided by these references is at least 25 bases, and may be as many as 400 or more bases in length. An important aspect of this reference is that the authors contemplate digesting longer dsRNAs to 21-25mer lengths with the endogenous nuclease complex that converts long dsRNAs to siRNAs in vivo. They do not describe or present data for synthesizing and using in vitro transcribed 21-25mer dsRNAs. No distinction is made between the expected properties of chemical or enzymatically synthesized dsRNA in its use in RNA interference.

Similarly, WO 00/44914, incorporated herein by reference, suggests that single strands of RNA can be produced enzymatically or by partial/total organic synthesis. Preferably, single stranded RNA is enzymatically synthesized from the PCR™ products of a DNA template, preferably a cloned cDNA template and the RNA product is a complete transcript of the cDNA, which may comprise hundreds of nucleotides. WO 01/36646, incorporated herein by reference, places no limitation upon the manner in which the siRNA is synthesized, providing that the RNA may be synthesized in vitro or in vivo, using manual and/or automated procedures. This reference also provides that in vitro synthesis may be chemical or enzymatic, for example using cloned RNA polymerase (e.g., T3, T7, SP6) for transcription of the endogenous DNA (or cDNA) template, or a mixture of both. Again, no distinction in the desirable properties for use in RNA interference is made between chemically or enzymatically synthesized siRNA.

U.S. Pat. No. 5,795,715 reports the simultaneous transcription of two complementary DNA sequence strands in a single reaction mixture, wherein the two transcripts are immediately hybridized. The templates used are preferably of between 40 and 100 base pairs, and which is equipped at each end with a promoter sequence. The templates are preferably attached to a solid surface. After transcription with RNA polymerase, the resulting dsRNA fragments may be used for detecting and/or assaying nucleic acid target sequences.

2. Pharmaceutical Compositions and Routes of Administration

Pharmaceutical compositions of the present invention comprise administering an effective amount of one or more inhibitors that inhibit or down-regulate the PRL-1 activity (and/or an additional agent) dissolved or dispersed in a pharmaceutically acceptable carrier to a subject. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that contains at least one PRL-1 inhibitor or additional active ingredient will be known to those of skill in the art in light of the present disclosure, and as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for. example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

A pharmaceutical composition of the present invention may comprise different types of carriers depending on whether it is to be administered in solid, liquid or aerosol form, and whether it needs to be sterile for such routes of administration as injection. A pharmaceutical composition of the present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the foregoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to a subject can be determined by physical and physiological factors such as body weight, severity of condition, the type of disease being treated, previous or concurrent therapeutic interventions, idiopathy of the patient and on the route of administration. The number of doses and the period of time over which the dose may be given may vary. The practitioner responsible for administration will, in any event, determine the concentration of active ingredient(s) in a composition and appropriate dose(s), as well as the length of time for administration for the individual subject.

In certain embodiments, pharmaceutical compositions may comprise, for example, at least about 0. 1% of an active compound. In other embodiments, the active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kglbody weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof

A PRL-1 inhibitor(s) of the present invention may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In certain aspects of the invention, the PRL-1 inhibitors are prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

F. Combination Therapies with PRL-1 Inhibitor(s)

In order to increase the effectiveness of a cancer treatment with the compositions of the present invention, such as a PRL-1 inhibitor, it may be desirable to combine these compositions with other cancer therapy agents. For example, the treatment of a cancer may be implemented with therapeutic agents of the present invention in conjunction with other anti-cancer therapies. Thus, in the present invention, it is contemplated that a PRL-1 inhibitor(s) may be used in conjunction with a chemotherapeutic, a radiotherapeutic, an immunotherapeutic or other biological intervention, in addition to pro-apoptotic or cell cycle regulating agents or protein tyrosine phosphatase regulators.

This process may involve contacting the cell(s) with a PRL-1 inhibitor and a therapeutic agent at the same time or within a period of time wherein separate administration of the inhibitor and an agent to a cell, tissue or organism produces a desired therapeutic benefit. The terms “contacted” and “exposed,” when applied to a cell, tissue or organism, are used herein to describe the process by which a PRL-1 inhibitor and/or therapeutic agent are delivered to a target cell, tissue or organism or are placed in direct juxtaposition with the target cell, tissue or organism. The cell, tissue or organism may be contacted (e.g., by administration) with a single composition or pharmacological formulation that includes both a PRL-1 inhibitor and one or more agents, or by contacting the cell with two or more distinct compositions or formulations, wherein one composition includes a PRL-1 inhibitor and the other includes one or more agents.

1. Regimens

The PRL-1 inhibitor may precede, be concurrent with and/or follow the other agent(s) by intervals ranging from minutes to weeks. In embodiments where the PRL-1 inhibitor and other agent(s) are applied separately to a cell, tissue or organism, one would generally ensure that a significant period of time did not expire between the time of each delivery, such that the inhibitor and agent(s) would still be able to exert an advantageously combined effect on the cell, tissue or organism. For example, in such instances, it is contemplated that one may contact the cell, tissue or organism with two, three, four or more modalities substantially simultaneously (i.e., within less than about a minute) as the inhibitor. In other aspects, one or more agents may be administered within of from substantially simultaneously, about 1 minute, about 5 minutes, about 10 minutes, about 20 minutes about 30 minutes, about 45 minutes, about 60 minutes, about 2 hours, or more hours, or about 1 day or more days, or about 4 weeks or more weeks, or about 3 months or more months, or about one or more years, and any range derivable therein, prior to and/or after administering the PRL-1 inhibitor.

Various combinations of a PRL-1 inhibitor(s) and a cancer therapeutic may be employed in the present invention, where a PRL-1 inhibitor is “A” and the secondary agent, such as a chemotherapeutic or radiotherapeutic agent, or any other cancer therapeutic agent is “B”:

A/B/A B/A/B B/B/A A/A/B A/B/B B/A/A B/B/B/A B/B/A/
B A/A/B/B A/B/A/B A/B/B/A B/B/A/A B/A/B/A B/A/A/B
A/A/A/B B/A/A/A A/B/A/A A/A/B/A A/B/B/B B/A/B/B

Administration of a PRL-1 inhibitor of the present invention to a patient will follow general protocols for the administration of that particular secondary therapy, taking into account the toxicity, if any, of the PRL-1 inhibitor treatment. It is expected that the treatment cycles would be repeated as necessary. The compositions employed ill the present invention may be administered once or more than once to a subject. It also is contemplated that various cancer therapies, such as chemotherapy, radiotherapy, as well as surgical intervention, may be applied in combination with the described pancreatic cancer therapy.

2. Anti-Cancer Therapies

An “anti-cancer” agent as contemplated for use with the present invention would be capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. Anti-cancer agents include biological agents (biotherapy), chemotherapy agents, and radiotherapy agents. The combination of chemotherapy with biological therapy is known as biochemotherapy.

In the present invention a composition that inhibits PRL-1 activity and an anti-cancer agent would be provided in a combined amount effective to kill or inhibit proliferation of the cell. This process may involve contacting the cells with the PRL-1 inhibitor and the agent(s) or factor(s) at the same time. This may be achieved by contacting the cell with a single composition or pharmacological formulation that includes both the PRL-1 inhibitor and the other agent, or by contacting the cell with two distinct compositions or formulations, at the same time, wherein one composition includes the PRL-1 inhibitor and the other includes the second agent(s).

a. Chemotherapy

It is also contemplated in the present invention a PRL-1 inhibitor(s) may be used in combination with chemotherapeutic agents. Such chemotherapeutic agents may include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, famesyl-protein transferase inhibitors, transplatinum, 5-fluorouracil, vincristine, vinblastine and methotrexate, Temazolomide (an aqueous form of DTIC), or any analog or derivative thereof. One example of a chemotherapuetuc agent currently used to treat pancreatic cancer is gemcitaben. Other studies employ high doses of 5-Fluorouracil (5-FU) for treatment of advanced pancreatic cancer.

The PRL-1 inhibitors may also be used in combination with other chemotherapeutic agents such as protein tyrosine kinase inhibitors. Such inhibitors may suitably include imatinib or imatinib mesylate (STI-571, Gleevec™; Norvartis, Inc.,), OSI-774 (Tarceva™; OSI Pharmaceuticals, Inc.,), ZD-1839 (Iressa®); AstraZeneca, Inc.,), SU-101 (Sugen, Inc.,) and CP-701 (Cephalon, Inc.,).

b. Radiotherapy

Another therapy that may be used in conjunction with a PRL-1 inhibitor(s) of the present invention to treat a cancer is radiotherapy. It is contemplated that radiotherapeutic factors that may be employed in the present invention are factors that cause DNA damage and have been used extensively, such as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the cancer or tumor cells.

c. Immunotherapy

The present invention also contemplates the use of immunotherapy in conjunction with a PRL-1 inhibitor(s). Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells. The combination of therapeutic modalities, i.e., inhibition or reduction of PRL-1 expression or activity would provide therapeutic benefit in the treatment of cancer, such as pancreatic cancer.

Immunotherapy could also be used as part of a combined therapy. The general approach for combined therapy is discussed herein. In one aspect of immunotherapy, the tumor cell must bear some marker that is amenable to targeting, i.e., is not present on the majority of other cells. Many tumor markers exist and any of these may be suitable for targeting in the context of the present invention. Common tumor markers which have been found to be upregulated in pancreatic cancer include, but are not limited to carcinoembryonic antigen, CA 27-29 antigen, neuron-specific enolase (NSE), CA 125 antigen, and human chorionic gonadotropin (HCG).

Other types of immunotherapy that may be employed with a PRL-1 inhibitor(s) of the present invention are passive and active immunotherapy.

A number of different approaches for passive immunotherapy of cancer exist. They may be broadly categorized into the following: injection of antibodies alone; injection of antibodies coupled to toxins or chemotherapeutic agents; injection of antibodies coupled to radioactive isotopes; injection of anti-idiotype antibodies; and finally, purging of tumor cells in bone marrow. It may be favorable to administer more than one monoclonal antibody directed against two different antigens or even antibodies with multiple antigen specificity. Treatment protocols also may include administration of lymphokines or other immune enhancers as described by Bajorin et al. (1988). The development of human monoclonal antibodies is well known to those of skill in the art (see Harlow and Lane, 1988)

In active immunotherapy, an antigenic peptide, polypeptide or protein, or an autologous or allogenic tumor cell composition or “vaccine” is administered, generally with a distinct bacterial adjuvant (Ravindranath and Morton, 1991; Mitchell et al., 1990; Mitchell et al., 1993).

d. Gene Therapy

The present invention also contemplates gene therapy in conjunction with PRL-1 inhibitor therapy. As with the majority of human cancers, numerous genetic alterations have been identified that play a role in adenocarcinoma of the pancreas. These include mutations in the tumor suppressor genes p53, Rb, p16, BRCA2 and DPC4. Several activated oncogenes have also been identified as contributing to pancreas cancer including K-ras, HER-2/neu, NFkappaB and AKT2. There are, no doubt, many other genetic defects that contribute to the onset and progression of pancreatic cancer and identifying these mutants and the specific consequences of the defects will lead to a better understanding of how to treat this disease. Gene therapy make also be combined with chemo- and radiotherapy to further improve the efficacy of the inhibitor of the present invention. For example, the herpes simplex-thymidine kinase (HS-tK) gene, when delivered to brain tumors by a retroviral vector system, successfully induced susceptibility to the antiviral agent ganciclovir (Culver et al., 1992).

Inhibitors of cell proliferation, such as tumor suppressor genes, may be employed with the PRL-1 inhibitor(s) of the present invention. The tumor suppressor oncogenes function to inhibit excessive cellular proliferation. The inactivation of these genes destroys their inhibitory activity, resulting in unregulated proliferation. The tumor suppressors p53, p16, Rb, and MMACl/PTEN may be employed with a PRL-1 inhibitor(s) of the present invention in treating a cancer, such as pancreatic cancer. Other genes that may be employed with a PRL-1 inhibitor of the present invention include APC, DCC, NF-1, NF-2, WT-1, MEN-I, MEN-II, zac1, p73, VHL, DBCCR-1, FCC, rsk-3, p27, p27/p16 fusions, p21/p27 fusions, anti-thrombotic genes (e.g., COX-1, TFPI), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC. These genes are provided herein as examples and are not meant to be limiting.

Genes that regulators of apoptosis, or programmed cell death, may also be employed with PRL-1 inhibitor(s) of the present invention in treating pancreatic cancer. Apoptosis, or programmed cell death, is an essential process for normal embryonic development, maintaining homeostasis in adult tissues, and suppressing carcinogenesis (Kerr et al., 1972). The Bcl-2 family of proteins have been demonstrated, in the art, to be important regulators and effectors of apoptosis in numerous systems. Some members of this family e.g., Bax, Bak, Bik, Bim, Bid, Bad, Harakiri, are known to promote cell death and thus may be employed with the PRL-1 inhibitor(s) of the present invention.

e. Hormonal Therapy

Hormonal therapy may also be used in conjunction with a PRL-1 inhibitor(s) of the present invention or in combination with any other cancer therapy described herein. The use of hormones may be employed to lower the level or block the effects of certain hormones that may play a role in the tumor cell proliferation. This treatment is often used in combination with at least one other cancer therapy as a treatment option or to reduce the risk of metastases in cancers which include but are not limited to breast, prostate, ovarian, or cervical cancer.

f. Surgery

The present invention may also be used in conjunction with surgery. Surgery may also be used in combination with any of the other cancer therapies described herein such as radiation therapy and chemotherapy.

Surgery may be used to remove all or part of the pancreas. The extent of surgery depends on the location and size of the tumor, the stage of the disease, and the patient's general health. Surgery may employ various procedures. One type of surgical procedure that may be use to treat pancreatic cancer is the Whipple procedure. In this procedure, if the tumor is in the head (the widest part) of the pancreas, the surgeon removes the head of the pancreas and part of the small intestine, bile duct, and stomach. The surgeon may also remove other nearby tissues. Another surgical procedure is a distal pancreatectomy in which the surgeon removes the body and tail of the pancreas if the tumor is in either of these parts. A total pancreatectomy may also be performed in which the surgeon removes the entire pancreas, part of the small intestine, a portion of the stomach, the common bile duct, the gallbladder, the spleen, and nearby lymph nodes.

### G. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Material and Methods

Microarray Sample Preparation and Hybridization. cDNA microarray slides used in this study were fabricated in the microarray core facilities at the Arizona Cancer Center (Calaluce et al., 2001). Briefly, each slide has 5760 spots divided into four blocks, with each containing eight identical ice plant genes from Mesembryanthemum crystallinum and 23 different housekeeping genes as references for data normalization. Each slide had 5289 unique human cDNA sequences. Poly(A)⁺ RNA was directly isolated from cell pellets using the FastTrack 2.0 kit (Invitrogen, Carlsbad, Calif.), following the instruction manual provided by the manufacturer. Normal pancreas Poly(A)⁺ RNA was isolated from total RNA, which was purchased from Clontech Laboratories (Palo Alto, Calif.) using the Oligotex Direct mRNA kit (Qiagen, Inc., Valencia, Calif.). This “normal pancreata” consisted of a pool of two tissue specimens donated by two male Caucasians 18 and 40 years of age. Labeling and purification of cDNA probes were carried out using the MICROMAX direct cDNA microarray system (NEN Life Science Products, Boston, Mass.). Two to 4 μg of the Poly(A)⁺ RNA samples were used for each labeling. Probes for each pancreatic cell line were labeled with cyanine 5 (Cy5), and probes for HeLa cells were labeled with cyanine 3 (Cy3). For HeLa cell versus normal pancreas hybridization, a normal pancreas sample was labeled with Cy3, and a HeLa cell sample was labeled with Cy5.

Purified cDNA probes were dried and dissolved in 15 μl of hybridization buffer (included in the MICROMAX direct cDNA microarray system kit). The probes were then denatured by heating at 95° C. for 2 min and applied to the array area of a predenatured microarray slide. The microarray slide was covered with a 22×22-cm slide coverslip and incubated in a HybChamber (GeneMachines, San Carlos, Calif.) at 62° C. for overnight. On the second day, the slide was washed in 0.5×SSC, 0.01% SDS for 5 min; 0.06×SSC, 0.01% SDS for 5 min; and 0.06×SSC for 2 min. Finally, the slide was dried by spinning at 500×g for 1 min and scanned in a dual-laser (635 nm for red fluorescent Cy5 and 532 nm for green fluorescent Cy3) microarray scanner (GenePix 4000; Axon Instruments, Foster City, Calif.).

RT-PCR. Two μg of total RNA isolated from pancreatic cancer cell pellets or frozen pancreatic tumor tissues were used for reverse transcriptase reactions (20 μl in total volume), which were carried out using the Omniscript RT kit (Qiagen, Inc.), following the manufacturer's protocol. The PCRs were then carried out by mixing 2 μl of reverse transcriptase reaction mixture, 5 μl of 10×PCR™ buffer containing 15 mM Mg²⁺, 1 μl of 10 mM deoxynucleotide triphosphate mixture, 2.5 μl of 5 μM PCR™ primer pair for specific gene, 1 μl of β-actin primer pair, 1 μl of β-actin competimers (Ambion, Inc., Austin, Tex.), 37 μl of H₂O, and 0.5 μl of 5 units/μl Taq polymerase (Promega Corp., Madison, Wis.). The amplification cycle (94° C. for 30 s; 56° C. for 45 s; and 72° C. for 1 min) was repeated 29 times. PCR™ primers for individual genes were designed to generate a DNA fragment ˜600 bp in length (if the mRNA itself is less than 600 bases, PCR™ products were generated in maximal length) using the Primer3 program (Rozen and Skaletsky, 2000).

Northern Blot. RNA electrophoresis and transferring to Zeta-Probe GT membranes (Bio-Rad, Hercules, Calif.) were performed as described previously (Calaluce et al., 2001). ³²P-labeled probes were made from the agarose gel-purified RT-PCR products of each gene using the RadPrime DNA Labeling System (Invitrogen). The probe hybridization and stripping buffers and conditions were as provided by the membrane manufacturer. Hybridized membranes were exposed to a Phosphorlmager (Molecular Dynamics, Sunnyvale, Calif.), and signals were quantified using the ImageQuant software.

Pancreatic Tumor Tissue Array Construction and Immunohistochemistry. Morphologically representative areas of 42 archival cases of pancreatic tumors, 35 of which are documented ductal adenocarcinomas, from the University of Arizona Health Sciences Center and the Tucson Veterans Administration Medical Center, are selected from formalin-fixed tissue samples embedded in paraffin blocks. Two 1.5-mm-diameter cores/case are reembedded in a tissue microarray using a tissue arrayer (Beecher Instruments, Silver Spring, Md.) according to a method described previously (Kononen et al., 1998). Ser. sections of the paraffin-embedded pancreatic tissue array are deparaffinized and reacted with primary antibodies specific for c-Myc (clone 9E10.3; Neo-Markers, Fremont, Calif.) or Rad51(Oncogene, Boston, Mass.). Before antibody incubation, the slides are processed for antigen retrieval. This consist of microwaving the slides in citrate buffer (0.1 M, pH 6.0) in a pressure cooker for 25 min and then leaving them to cool. The slides will be incubated with the antibody for 1 h. Biotinylated anti-mouse/anti-rabbit secondary antibodies are applied, followed by streptavidin-peroxidase complex (DAKO, Carpinteria, Calif.). Colored products are produced using the diaminobenzidine substrate. Staining reactions are scored as diffuse or focal and graded (from 0, negative to 4+, intensely positive) for both neoplasm and background stroma.

Antisense Experiments. To perform antisense experiments cells (5×10⁵) are incubated in triplicate in 6-well plates in 1 ml of culture medium supplemented with 10% heat-inactivated FCS for 30 min at 65° C. to destroy nuclease activity. These cells are then cultured (24 h) in the presence or absence of antisense, sense, or randomly scrambled phosphorothioate oligonucleotides (ODNs). The ODN sequences are tested against sequences in the GenBank™ database. Two distinct antisense ODNs complementary to sequences that encompass the translation initiation site of the specific target are used. The ability of the antisense to inhibit expression of the target is verified by RT-PCR and with an antibody for Western blotting. Once it is confirmed that expression of the target is downregulated, the phenotypic consequences of inhibition of the target can be determined using the assays describe below.

Cell Proliferation Assays. Cell proliferation assays with these cell lines are performed to determine the effect of target inhibition on cell growth. Cells are seeded at 2.0-5.0×10⁵ cells in 100-mm culture dishes and allow to attach overnight at 37° C. Adherent cells are washed and incubated with serum-free RPMI 1640 or RPMI containing 10% FBS for 48 h, after which they are trypsinized and counted using a hemocytometer. In addition, parallel experiments are perform and instead of cell counts, the proliferative status of the cell lines is determined using flow cytometric analysis of DNA content. For flow analysis, cells are stained with propidium iodide using a modified Krishan technique (Krishan, 1975). All samples are analyze with an FACSCAN flow cytometer (Becton Dickinson) using a 15 mWatt argon ion laser operated at 6 mWatts of power at 488 nm. Photomultiplier tube voltage is adjusted for each control sample to position the G0/G1 to channel 240 on a 1024 channel presentation. Histograms are analyze for cell cycle compartments using CELLQUEST (Becton Dickinson) analysis software. Histograms having 50K events are collected to maximize the statistical validity of the compartmental analysis. The results of this flow analysis allow the examination of the cell cycle distribution of the pancreatic cancer cell lines.

Alternative analysis of proliferative rate can be estimated by a number of other techniques, including BrdU incorporation or PCNA or Ki67 immunostaining, however, flow analysis is preferred, since it provides an estimate of the fraction of cells in the G1 and G2/M stages of the cell cycle as well as in S-phase. By measuring the proliferative status of the cell lines a better understanding of whether or not the target plays a role in regulating the growth of pancreatic cancer cells is achieved.

Apoptosis Assays. For the measurement of spontaneous and serum starvation induced apoptosis before and after target inhibition, the cells are seeded at 2.0-5.0×10⁵ cells in 100-mm culture dishes and allow to attach overnight at 37° C. Adherent cells are washed and incubated with serum-free RPMI 1640 media or RPMI 1640 media containing 10% FBS for 48 hr, at which time they are harvested by trypsinization. Any floating cells in the media will be saved and pooled with the harvested cells for the apoptosis analysis. An annexin V based assay is used to quantitate apoptosis. After initiating apoptosis, cells translocate phosphatidylserine (PS) from the inner face of the plasma membrane to the cell surface. Once on the cell surface, PS can be detected using a GFP or FITC conjugate of annexin V (Clontech), a protein that has a strong, natural affinity for PS. This simple assay is a one-step staining procedure of live cells that takes 10 minutes. After incubation with the conjugated annexin V the cells are analyzed by flow cytometry and the percentage of labeled cells determined.

Anchorage Dependent Cell Growth. To assess anchorage-independent growth, the target inhibited cells are suspended in reduced-serum (2%) medium containing 0.3% agar, and overlaid onto a 0.6% agar base at a density of 2×10⁴ cells/60-mm dish. Colony formation is monitored for up to 1 month. The number of colonies formed by the target inhibited and uninhibited cells is counted and compared for statistical differences. The ability of target inhibition to alter anchorage-independency of the pancreatic cancer cell lines is a good indication of whether the target is involved in promoting tumorigenicity in pancreatic cancer cell lines.

Cell Migration. In addition to anchorage-independent cell growth, the role of target inhibition in suppressing cell migration can be assessed. Multiple signaling pathways are believed to play a role in directed cell migration. Cell migration is assessed by quantitating the number of cells that directionally migrate through membranes to a collagen undercoating. Briefly, 1× target inhibited and uninhibited cells are loaded into modified Boyden chambers (tissue culture—treated, 6.5-mm diameter, 10 μm thickness, 8-μm pores, Transwell®; Costar Corp) containing collagen type I-undercoated membranes. Cells are allowed to migrate through membranes by incubating them at 37° C. for various time points. Nonmigratory cells on the upper membrane surface are removed with a cotton swab, and the migratory cells attached to the bottom surface of the membrane are stained with 0.1% crystal violet in 0.1 M borate, pH 9.0, and 2% ethanol for 20 min at room temperature. The number of migratory cells per membrane is either counted with an inverted microscope using a 40× objective, or the stain is eluted with 10% acetic acid and the absorbance at 600 nm determined and migration is enumerated from a standard curve. Differences in the migration capacity of cells between target inhibited and uninhibited cells is evaluated by comparing the percentages. A decrease in the migration capacity indicates that the target plays a role in regulating cell invasiveness.

Analysis of Gene Expression Patterns. From frozen pancreatic cancer specimens, frozen tissue sections can be made and examined independently of the original pathological report. Total RNA is extracted, using the standard Triazol RNA isolation protocol (Life Technologies, Gaithersburg, Md.), from tissue blocks that contained over 75% of neoplastic cells. The amount and the quality of RNA is checked by electrophoresis on a 1% formamide agarose gel. Normal tissue RNA samples can be obtained from Clontech (Palo Alto, Calif.). The RNA is labeled by reverse transcription and array hybridizations to the new 10,000-gene chip is performed as described above. After analysis, gene expression patterns from the frozen tissue are compared to those from the cell lines to look for significant differences and for potential new targets.

### Example 2

### Microarray Analysis

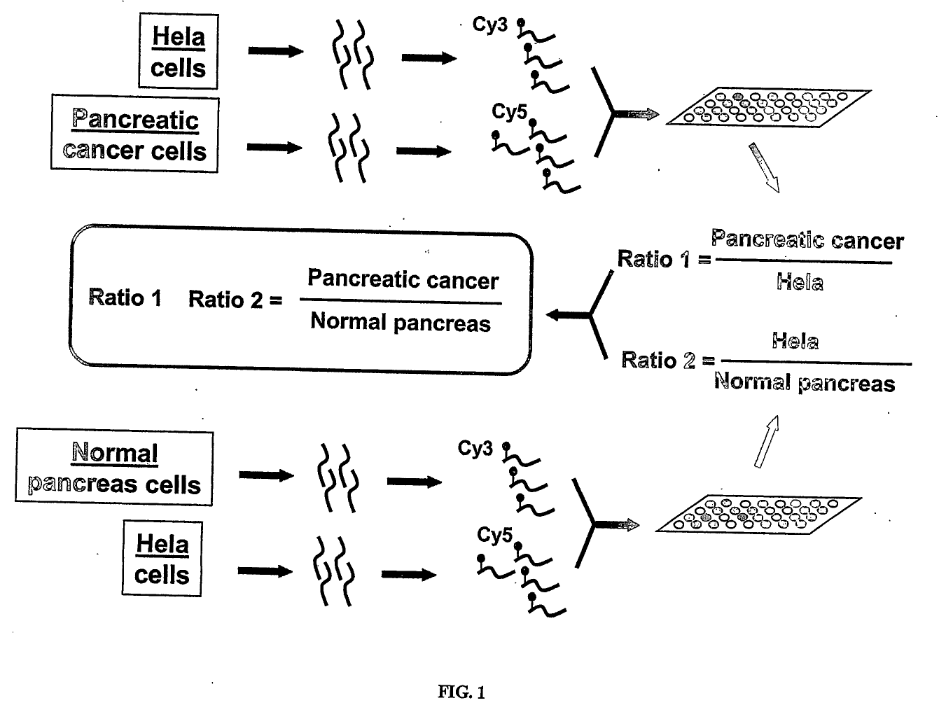

The gene expression patterns of genes from pancreatic cancer cell lines were analyzed and compared to gene expression in normal pancreas cells. The strategy employed is shown in FIG. 1. Instead of performing straight comparisons of gene expression in the pancreatic cancer cell lines to normal pancreas, a universal reference RNA (Hela cell RNA) was used to hybridize to both the cancer cell lines and normal pancreas. The gene expression ratios were then calculated by dividing out the ratio data from the reference as shown in FIG. 1. The reference was used in this analysis because it allows for a comparison of multiple hybridizations when the control RNA (normal pancreas) is limiting.

### Example 3

### Hybridization of Expression Products from BXPC-3 Pancreatic Cancer Cells

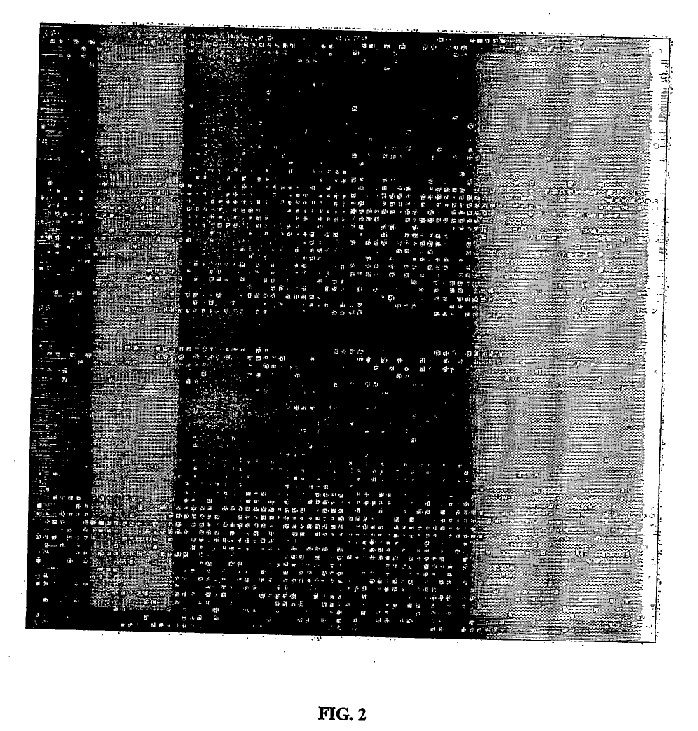

FIG. 2 shows a representative array hybridization from a pancreatic cancer cell line hybridized to a reference RNA. To date, gene expression patterns from several different pancreatic cancer cell lines have been analyzed, and compared to the gene expression patterns of normal pancreas. The probes for the cDNA microarray analysis were made using a fluorescent first strand cDNA from 4 μg Poly A+RNA from each of the pancreatic cancer cells in the presence of Cy5-dCTP (Red), and from 4 μg of Poly A+RNA from Hela cells in the presence of Cy3-dCTP (Green). The two fluorescent first strand cDNAs were then mixed, denatured, and used as targets for the genes on the cDNA microarray slide. Following the hybridization and wash steps, quantitative fluorescent emissions were collected using a Gene Pix 4000A-microarray reader (Axon Instrunents) and quantitated using the Gene Pix 4000A associated software. The normal pancreas RNA (purchased from Clontech and consisted of pooled RNA from 4 different donors) was analyzed in the same way with the Cy5 channel being Hela cell RNA and Cy3 channel being normal pancreas. Each cell line was then compared to normal pancreas by simply multiplying the ratio data from the cell lines to the normal pancreas. The gene expression ratio data for each gene was then analyzed and genes showing significant changes in gene expression were identified using a 95% confidence interval analysis. Hierarchical cluster analysis was then used to cluster genes with similar expression patterns into groups. From this analysis, 438 genes were identified as being significantly downregulated across the pancreas cancer cell lines and 68 genes were significantly upregulated. The 68 upregulated genes were screened further for suitability as drug targets. A list of 50 of these overexpressed genes, including both known and unknown genes, is shown in Table 1. Examples of potential targets identified by the cDNA microarray include protein tyrosine phosphatase 1 (PRL-1), urokinase type-plasminogen activator (uPA) and its receptor (uPAR), aurora kinase, CDC28 protein kinase 2, CDC25B and 5′-nucleotidase.

TABLE 1
50 Overexpressed Genes In Pancreatic Cancer Tissues

| | |
|---|---|
| EphA2 | Urokinase plasminogen activator |
| Non-specific cross reacting | PRL-1 phosphatase |
| antigen | |
| Thymosin beta | PCNA |
| Annexin I (lipocortin I) | Human Gu protein |
| MMP-9 | High mobility group (nonhistone) |
| Heparin cofactor II | CksHs-2 |
| Glutathione peroxidase 2 | Dysferlin |
| EVI2A | Trinucleotide repeat |
| | containing 3 GAGH3 |
| ESTs | HYPOTHETICAL PROTEIN |
| | KIAA0195 |
| Pho GTPase-specific | Small nuclear |
| GTP exchange factor | ribonucleoprotein IL1A |
| c-Myc | S-100P PROTEIN |
| Cytochrome c-1 | NGAL |
| NUCLEOLYSIN TIA-1 | Calgizzarin |
| Human small proline rich | Thioredoxin reductase |
| protein | |
| Aurora Kinsase 2 | Rho GDP dissociation |
| | inhibitor (GDI) beta |
| p33 ING1 | DNA primase polypeptide 1 (49 kD) |
| Annexin VIII | CCAAT/enhancer binding |
| | protein (C/EBP), beta |
| TROPONIN T | RAD51 |
| Single-stranded DNA- | Met receptor |
| binding protein | |
| Keratin 19 | NGF |
| Interferon consensus | Ciao-1 |
| sequence binding protein 1 | |
| Leman coiled-coil protein | EST R23055 |
| FOS-like antigen-1 | EST R06944 |
| TRANSCRIPTION | EST R53421 |
| ELONGATION FACTOR SH | |
| Plasminogen activator | EST R35245 |
| receptor, urokinase receptor | |

### Example 4

### Overexpression of PRL-1 in Pancreatic Cell Lines and Tumors

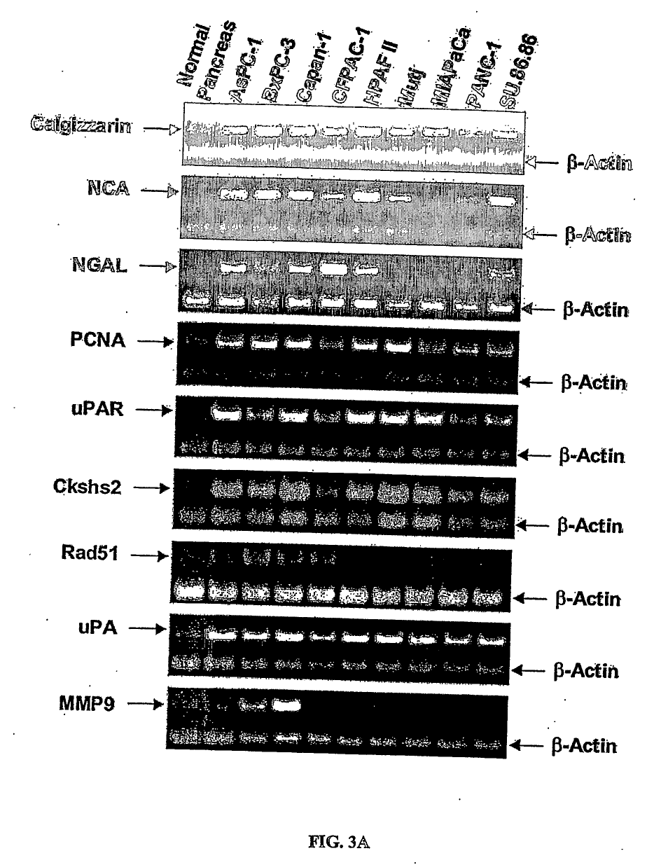

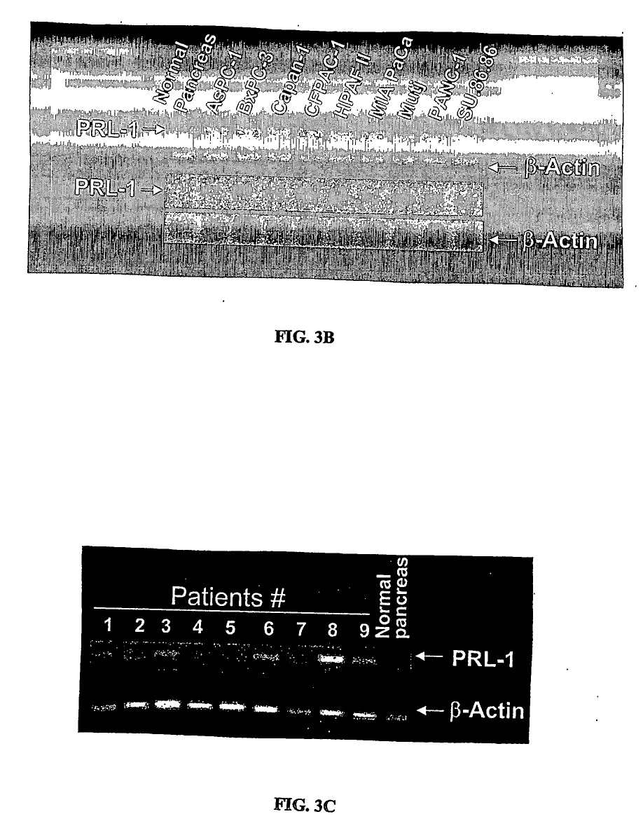

To further analyze and validate the expression patterns of these overexpressed genes in pancreatic cancer cell lines, RT-PCR and Northern blotting were used to look at each gene individually. FIG. 3A shows representative data from RT-PCR analysis of some of the genes that are overexpressed in pancreatic cancer cells versus normal pancreas.

PRL-1 was found to be one of genes that showed the most consistent and significant overexpression (Table 2; FIG. 3B). PRL-1 is overexpressed in 6 cell lines with a ratio ranging from 3.3 to 9.5. The other 3 cell lines did not have an expression ratio recorded because their microarray hybridization did not pass the quality control. FIG. 3C shows overexpression of PRL-1 in several patient tumor samples as compared to normal pancreas. The results from the RT-PCR and Northern blotting have confirmed overexpression of the genes from the microarray analysis and both sets of data correlate well with respect to differences in levels of expression across the different cell lines.

TABLE 2

| | Expression ratio |
|---|---|
| Cell Line | (Cell Line/Normal) |
| AsPC-1 | 4.9 |
| BxPC-3 | 5.6 |
| Capan-1 | N/A |
| CFPAC | N/A |
| Su86.86 | N/A |
| HPAF II | 9.5 |
| Mia Paca-2 | 4.5 |
| Mutj | 3.3 |
| Panc-1 | 3.7 |

### Example 5

### Pancreatic Cancer Tissue Array

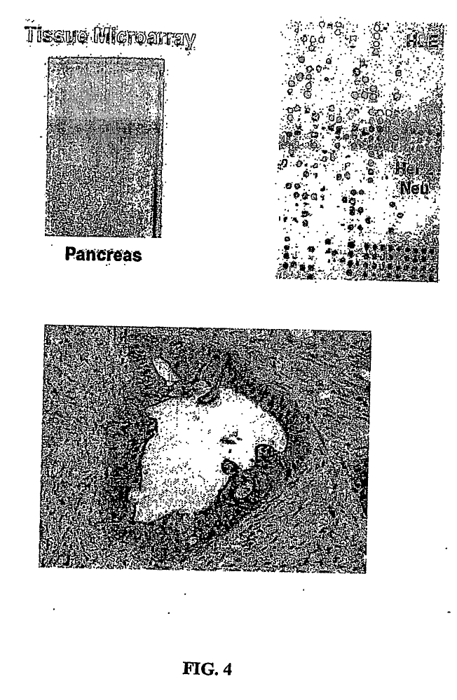

In addition to confirming overexpression of the target genes in the pancreatic cancer cell lines, a tissue array was developed that allows the determination of the expression of specific gene products in tumors taken from pancreatic cancer patients (see FIG. 4). The sampling of the original pancreatic cancer tissues for arraying was performed from morphologically representative regions of formalin-fixed paraffin-embedded tumor and normal tissue blocks. Core tissue biopsies (diameter 0.6 mm, height 3-4 mm) were taken from individual “donor” blocks and arrayed into a new “recipient” paraffin block (45×20 mm) using a tissue microarraying instrument (Beecher Instruments). On average, 200 sections can be cut from one tumor tissue microarray block. HE-staining for histology verification is performed on every 50th section cut from the block (FIG. 4). Once constructed, the tissue microarray slide was then stained using immunohistochemistry with antibodies directed against the proteins of interest and evaluated either manually or utilizing a high-throughput digital imaging system. This tissue array system greatly enhances the ability to quickly validate the expression of potential target genes and analyze the frequency of expression across a number of patient tumors.

### Example 6

### Effect of Antisense PRL-1 on Inhibiting Pancreatic Cancer Cell Growth

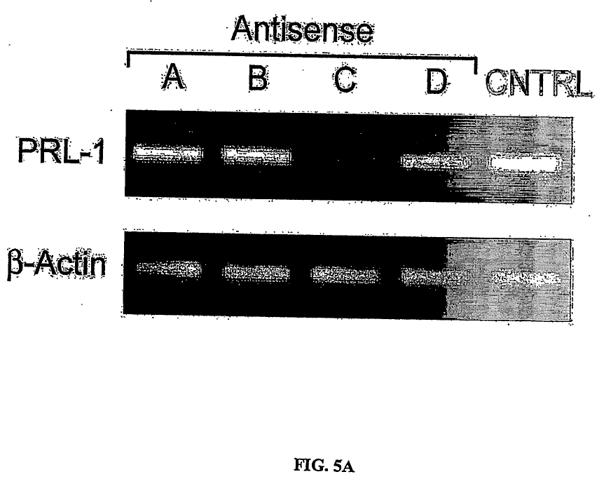

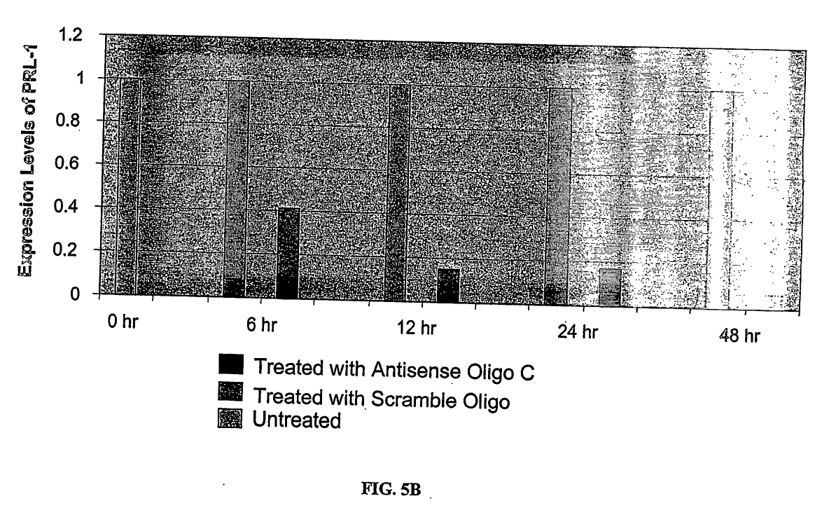

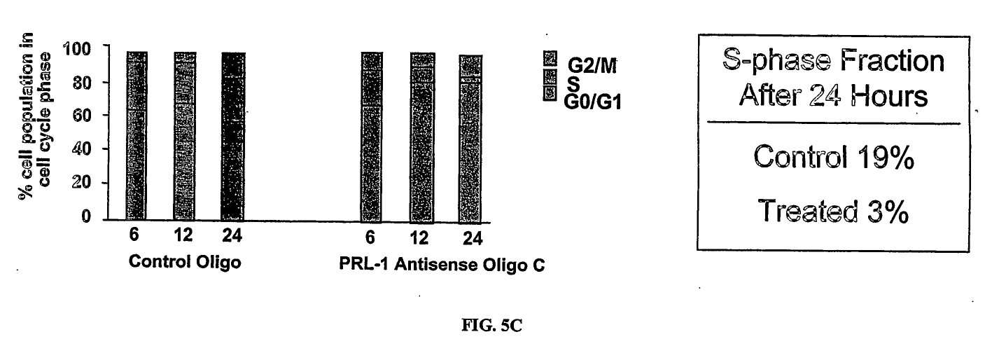

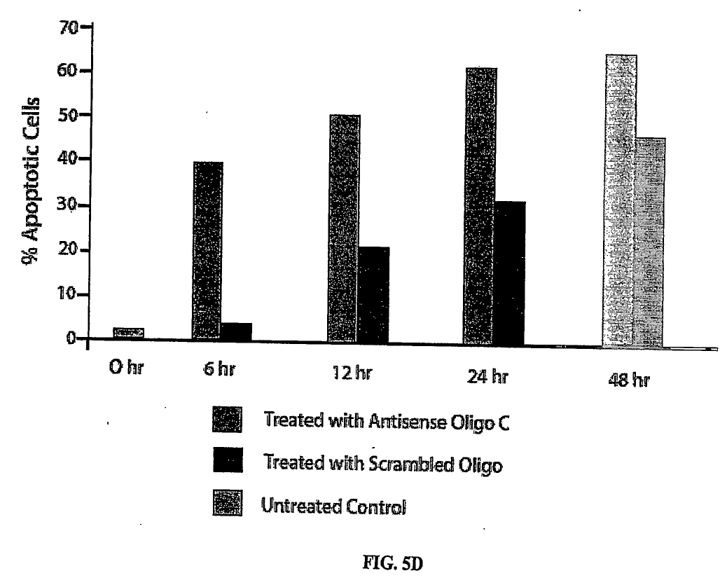

To investigate the effect of PRL-1 inhibition on pancreatic cancer cell growth, antisense oligonucleotide studies were conducted. Four antisense oligonucleotides were designed to target different areas of the PRL-1 mRNA. One of these oligonucleofides, AS-Prl-1C, reduced the mRNA level more than 90% within 24 hours of treatment (FIG. 5A) and, therefore, was chosen to be used in subsequent studies. A time course treatment of Mia PaCa-2 cells with 200 nM of AS-Prl-1C or the corresponding scramble was conducted and changes in PRL-1 mRNA level, cell cycle distribution and apoptosis population were examined. The PRL-1 mRNA level was reduced to its lowest level (˜5% of the control) 24 hours after the AS-Prl-1C treatment (FIG. 5B). The treatment of Mia PaCa-2 cells with PRL-1 antisense oligonucleotides resulted in arrest of cell growth in the G0/G1 phase of the cell cycle. Twenty-four hours after treatment, 85% of the cells were in G0/G1 phase and 3% of the cells were in S phase compared to 60% in G0/G1 and 19% in S phase for the scramble oligonucleotide treated samples (FIG. 5C). PRL-1 antisense oligonucleotide treatment also induced apoptosis in the Mia PaCa-2 cells. Twelve hours after AS-Prl-1C treatment, the number of apoptotic cells increased dramatically to 40% compared to less than 5% in scramble oligonucleotide control (FIG. 5D). These results indicated that PRL-1 plays a role in cell cycle regulation and that inhibition of PRL-1 induces apoptosis in tumor cells.

### Example 7

### Effect of siRNA Complexes on Inhibiting Pancreatic Cancer Cell Growth

Short interfering RNA (siRNA) has also been used to suppress PRL-1 expression in pancreatic cancer cells. In the present invention, double stranded siRNA complexes are designed using the following guidelines: (1) a double stranded RNA complex is composed of a 21-nucleotide sense and 21-nucleotide anti-sense strand, both with a 2-nucleotide 3′ overhang, i.e., a 19 nucleotide complementary region; (2) a 21 nucleotide sequence is chosen in the coding region of the mRNA with a G:C ratio as close to 50% as possible, preferably within about 60% to about 40%, or alternatively within about 70% to about 30%; (3) preferably regions within about 75 nucleotides of the AUG start codon or within about 75 nucleotides of the termination codon are avoided; (4) preferably more than three guanosines in a row are avoided as poly G sequences can hyperstack and agglomerate; (5) preferably choose a sequence that starts with AA as this results in siRNA's with dTdT overhangs that are potentially more resistant to nucleases; (6) preferably the sequence is not homologous to other genes to prevent silencing of unwanted genes with a similar sequence. A negative control may be included, such a negative control suitably being the same nucleotide sequence as the test siRNA but scrambled such that it lacks homology to any other gene.

Examples of such 21 nucleotide target DNA sequences, and the 19 nucleotide sense and antisense sequences utilizing dTdT 3′ overhangs (dT is 2′-deoxythymidine), derived from the coding sequence of PRL-1 (derived from GenBank NM_{—}003463[gi:17986281]), include, but are not limited to, those described in Table 3:

TABLE 3

| | | |
|---|---|---|
| Target RNA | Sense RNA | Antisense RNA |

| | | |
|---|---|---|
| aaacaaauuuauagaggaacu | Acaaauuuauagaggaacutt | aguuccucuauaaauuugutt |
| (SEQ ID NO:1) | (SEQ ID NO:2) | (SEQ ID NO:3) |
| aacaaauuuauagaggaacuu | Caauuuauagaggaacuutt | aaguuccucuauaaauuugtt |
| (SEQ ID NO:4) | (SEQ ID NO:5) | (SEQ ID NO:6) |
| aaagaagguauccauguucuu | Agaagguauccauguucuutt | aagaacauggauaccuucutt |
| (SEQ ID NO:7) | (SEQ ID NO:8) | (SEQ ID NO:9) |
| aaauacgaagaugcaguacaa | Auacgaagaugcaguacaatt | uuguacugcaucuucguautt |
| (SEQ ID NO:10) | (SEQ ID NO:11) | (SEQ ID NO:12) |
| aagaugcaguacaauucauaa | Gaugcaguacaauucauaatt | uuaugaauuguacugcauctt |
| (SEQ ID NO:13) | (SEQ ID NO:14) | (SEQ ID NO:15) |
| aauucauaagacaaaagcggc | Uucauaagacaaaagcggctt | gccgcuuuugucuuaugaatt |
| (SEQ ID NO:16) | (SEQ ID NO:17) | (SEQ ID NO:18) |

The orientation of the double stranded RNA complex for the first exemplified sense and antisense siRNA strands in Table 3 is as follows:

| | |
|---|---|
| 5′-acaaauuuauagaggaacutt-3′ | (SEQ ID NO:2) |
| 3′-ttuguuuaaauaucuccuuga-5′ | (SEQ ID NO:19) |

The above guidelines are solely an aid to designing suitable RNA oligonucleotides and are not a limitation of the interfering RNA oligonucleotides and related methods of use of the present invention.

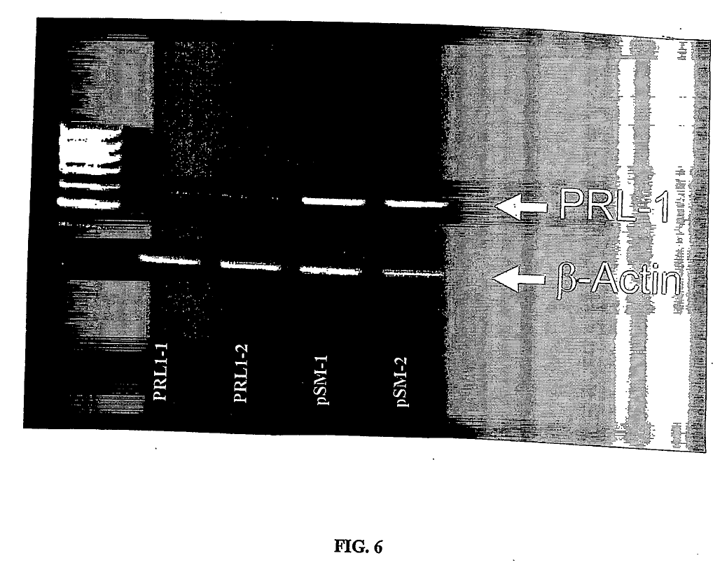

Thus far, two siRNA sequences have been identified that are able to reduce PRL-1 expression to about 10% of non-treated control (FIG. 6). The phenotypes of the PRL-1 siRNA treated cells are currently under examination, but it is predicted that the effects of siRNA will be similar to that seen with PRL-1 antisense oligonucleotides.

### Example 8

### Screening of Compound Libraries for PRL-1 Inhibitors Using An Enzymatic Assay

Two different but complementary approaches have been applied to identify novel small molecular weight inhibitors of PRL-1. One is the high throughput screen of small molecule libraries using an in vitro enzymatic assay of PRL-1. The other is the PRL-1 homolog model, based on virtual screening of chemical structure libraries and optimization of lead compounds.

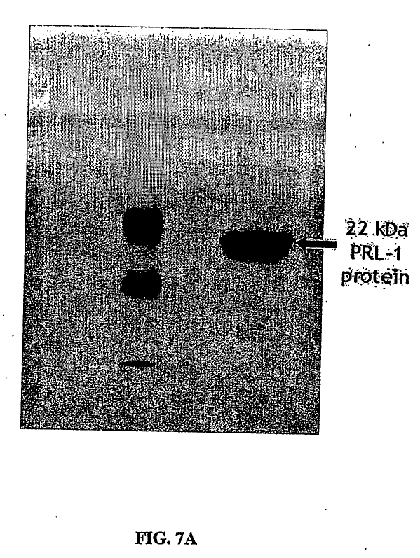

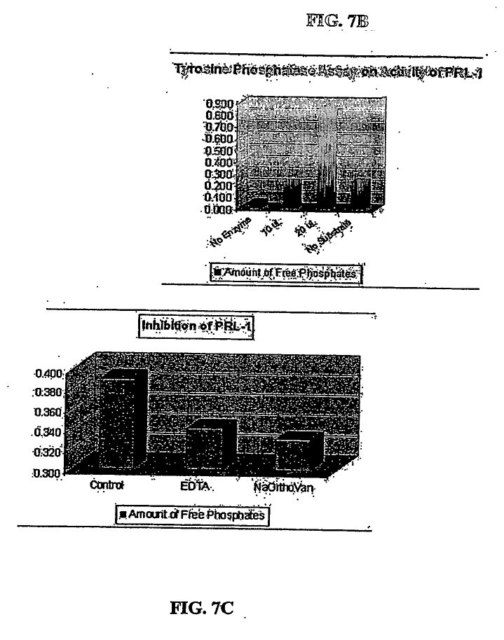

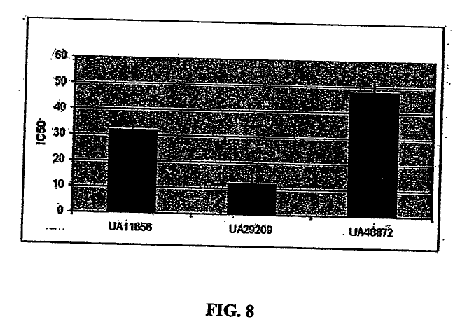

To produce PRL-1 protein for in vitro phosphatase assays, the PRL-1 gene was cloned into an expression vector with a His tag (pcDNA™ 3.1 from Invitrogen) and the protein expressed in vitro using the TNT coupled transcription and translation kit (Promega). FIG. 7A shows the Western blot detection of the His-tagged PRL-1 protein in the TNT mixture. The molecular weight of this PRL-1 protein (22KDa) is exactly the same as reported in the literature. An in vitro phosphatase assay was conducted using a tyrosine phosphatase assay system (Promega) to confirm the dephosphorylation activity of the recombinant PRL-1 protein. As shown in FIG. 7B, 20 μl of the dialyzed TNT product increased the phosphatase activity by 3 times compared to the control (from 0.2 to 0.8 in absorbance units). Two known tyrosine phosphatase inhibitors, sodium orthovanadate and EDTA, showed some inhibitory activity against PRL-1 (FIG. 7C).

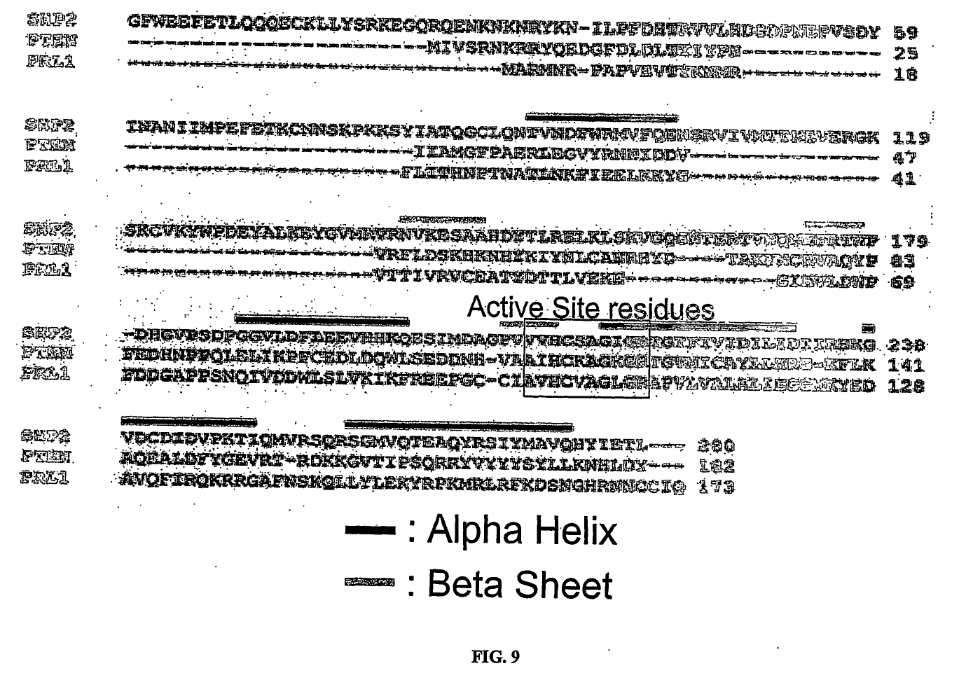

This assay was optimized and used to screen various compound libraries for PRL-1 inhibitors. FIG. 8 shows the anti-PRL-1 activity of some positive hits identified from the NCI diversity library and/or the University of Arizona (UA) Natural Products Library. Compounds identified using the DiFMUP in vitro assay to screen compounds from the Nanosyn Combichem library and the NCI database include NS19999, NS45609, NS45336, and NCI668394 respectively.

### Example 9

### Molecular Modeling and Virtual Screening of Chemical Structure Libraries

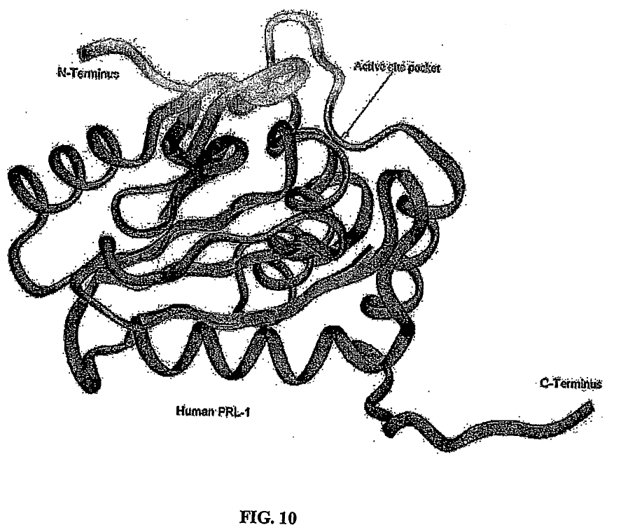

The three-dimensional structure of PRL-1 has not been solved yet. However, structures of various other phosphatases have been published. Given that PRL-1 has about 70% in catalytic domain and 21% overall sequence identity to PTEN, a lipid phosphatase, (FIG. 9) a homology model was built based on the PTEN crystal structure (FIG. 10).

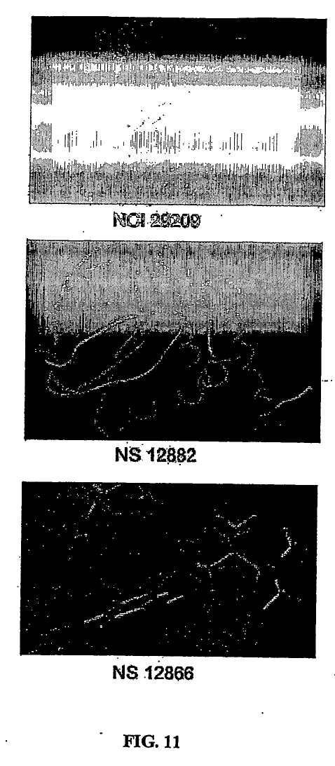

Based on this model compound structures from different sources have been docked to the active site. These structures include the NCI chemical database, known drug leads for the protein tyrosine phosphatases (PTPs) and inhibitors of the Cdc25B, a dual specfic phosphatase. A molecular spreadsheet was built within the Sybyl. Initially three separate databases were generated for input to the virtual docking. From the combined database a total of 49 structurally diverse compounds were obtained for further screening. The docking models of three compounds that have the best docking to the active site of PRL-1 are shown in FIG. 11 (the structures of these compounds are provided in table 5).

The NCI29209 is a substituted 6-methoxy-quinoline class of compound identified as PRL-1 inhibitor from high-throughput screening and molecular modeling methods. The NC129209 compound was obtained from an NCI database and tested on the NCI panel of cell lines for various cancers. This compound is being utilized as a lead compound for optimization for design of a novel series of compounds as PRL-1 inhibitors. Table 4 also shows a novel series of PRL-1 inhibitors designed using the structure based approach. These compounds are been analyzed using enzymatic and cellular assays.

The Nanosyn Combichem library compounds NS12866: [3-(Benzo[1,2,5]-4-sulfonyl-thiadiazole, NS12882: 2-Amino-4-trifluoromethanesulfonyl-benzoic acid, as shown in table 5, were modeled using a homology model of PRL-1. Based on FlexX docking the binding mode of these compounds have been explored and a novel series of compounds using a structure-based design strategy are been designed (Table 4). Table 5 also provides a list of PRL-1 inhibitors identified by screening various library as well as the IC₅₀ values which for some compounds was found to be >100 μM. As discussed, several compounds were identified by screening the NCI database and the Nanosyn Combichem library however, no hits were obtained from the screening of the LeadQuest (Tripos Inc.) and the MayBridge libraries.

TABLE 4
Novel PRl-1 Inhibitors

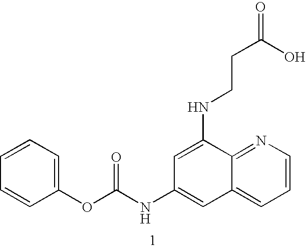

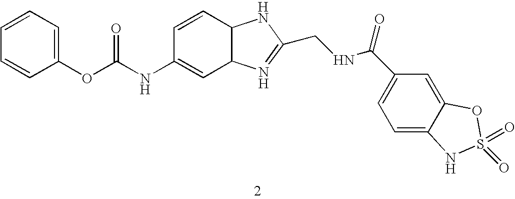

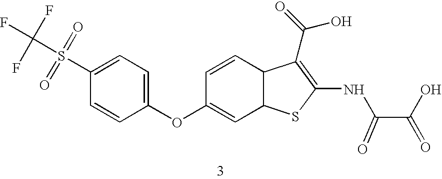

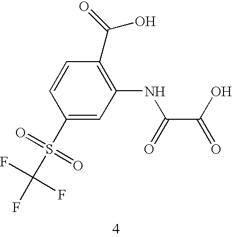

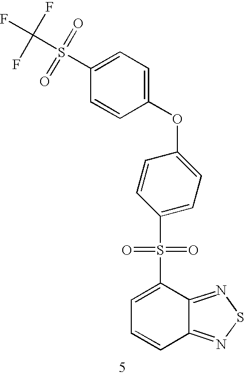

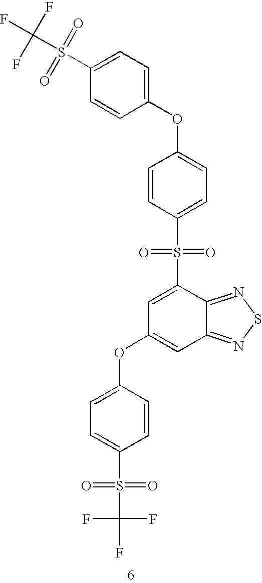

TABLE 5

| | | DOCKING | |
|---|---|---|---|
| STRUCTURE | NAME | VALUE | IC₅₀ |
| 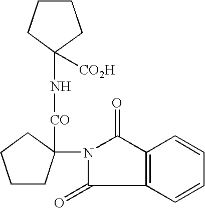 | UA78871 | −11.4 | N/A |
| 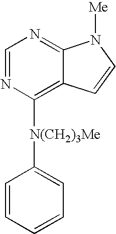 | UA11656 | −4.1 | — |
| 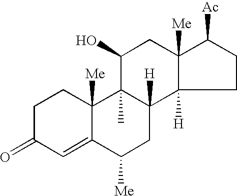 | UA53892 | −7.9 | N/A |
| 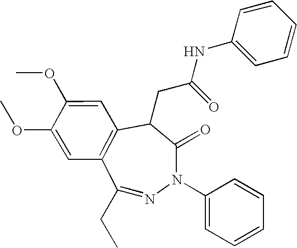 | UA12812 | −14.6 | 66 μM |
| 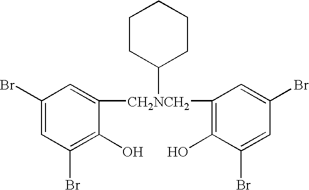 | UA48872 | −1.6 | N/A |
| 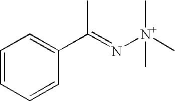 | UA97885 | 0 | N/A |
| 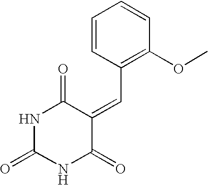 | UA12499 | −14.7 | 36.5 μM |
| 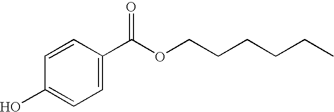 | UA12690 | −8.5 | N/A |
| 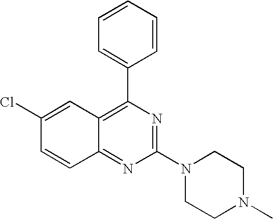 | UA13066 | −4.5 | — |
| 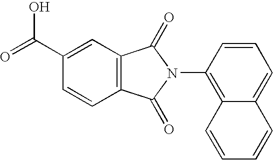 | UA13464 | −15.6 | 72 μM |
| 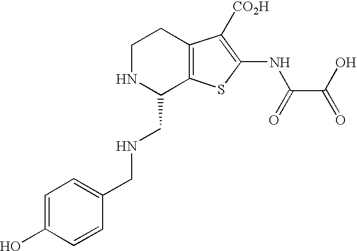 | PTP1B Nova Nordisk NNC-52-1236 | −26.2 | N/A |
| 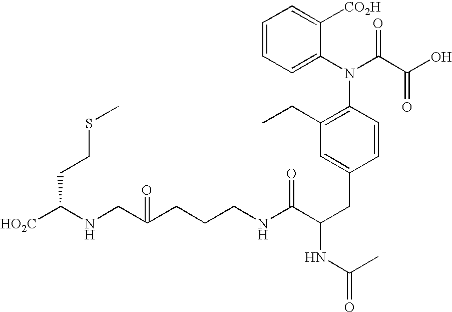 | Abbott-10 A-366901 | −19.8 | N/A |
| 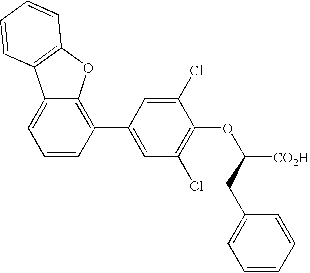 | Abbott A-321842 | −14.0 | N/A |
| 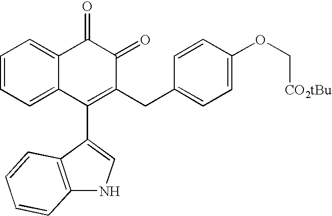 | Korea Research Institute of Chem. Tech. 1,2-naphtoquinone derivatives | −8.9 | N/A |
| 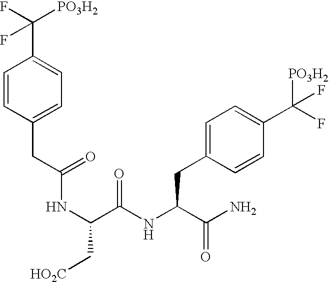 | Albert Einstein College of Med. 4′-phosphonyldifluoro methyl-phenylanaline derivatives | −13.9 | N/A |
| 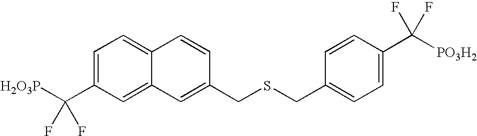 | Merck-Frosst methylphosphonc acid derivatives | −14.0 | N/A |
| 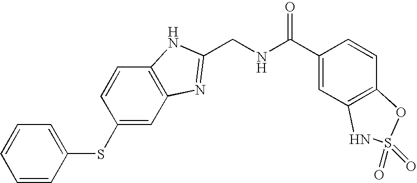 | Aventis Benzooxathiazole derivatives | −20.8 | N/A |
| 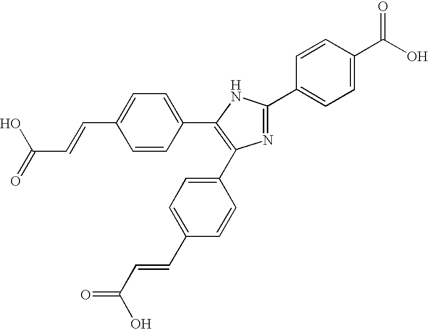 | Ontogen Cinnamic acid derivatives | −20.1 | N/A |
| 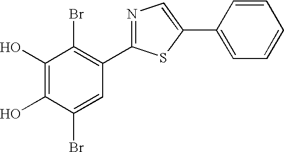 | Japan Tobacco Hydroxyphenyl azole derivatives | −8.4 | N/A |
| 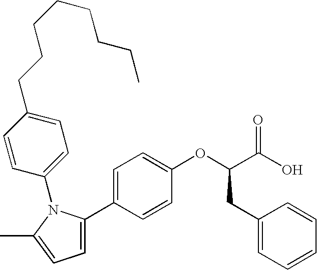 | Takeda Pyrrol phenoxy propionic acid derivatives | −8.6 | N/A |
| 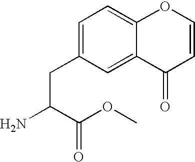 | Molecumetics Phenylatanine derivatives | −13.6 | N/A |
| 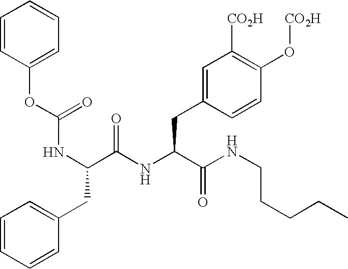 | Pharmacia 3′-caxboxy-4′ (O- carboxymethyl)- tyrosine derivatives | −27.7 | N/A |
| 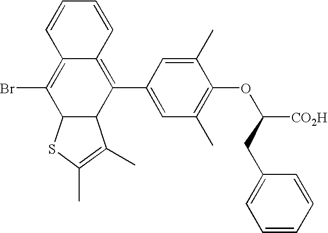 | Wyeth Ertiprotafib (Phase II discontinued) | −16.0 | N/A |
| 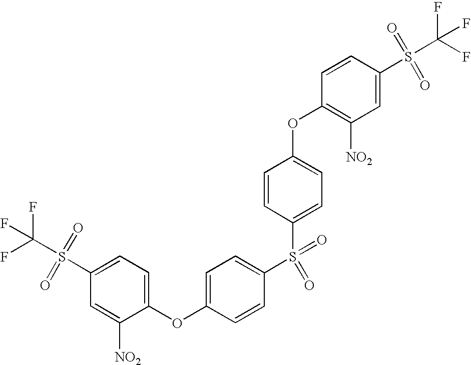 | Sugen Trifluoramethyl sulfonyl derivatives | −24.0 | N/A |
| 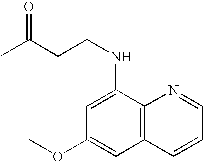 | UA292O9 and/or NCI29209 | −14.2 | 41 μM |
| 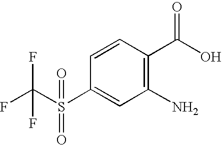 | UA12882 and/or NS12882 | −22.9 | 25 μM |
| 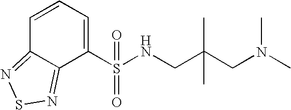 | UA12866 and/or NS12866 | −23.6 | N/A |
| 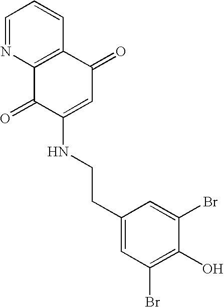 | UA668394 and/or NC1668394 | −11.2 | 7.2 μM |
| 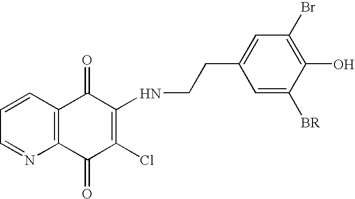 | UA668394-1 | | |
| 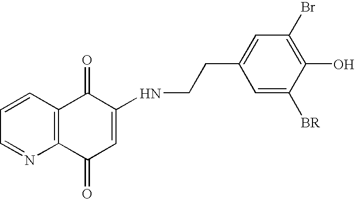 | UA668394-2 | | |
| 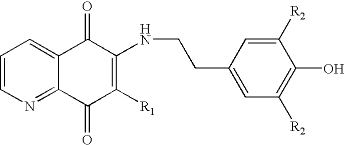 | R₁ = H, F, Cl R₂ = F, Cl, Br | | |
| 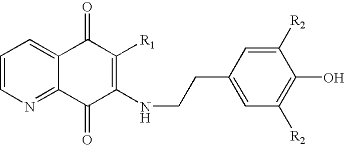 | R₁ = H, F, Cl R₂ = F, Cl, Br | | |
| 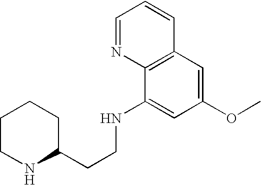 | UA13378 R/S | −14.1 | N/A |
| 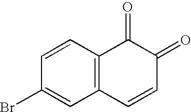 | UA13082 | −14.4 | 8.7 μM |
| 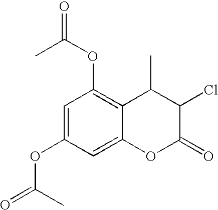 | UA14798 | −8.5 | 64 μM |
| 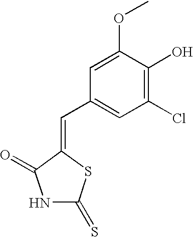 | UA16551 | −11.0 | N/A |
| 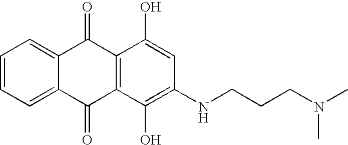 | UA339585 | −8.8 | N/A |
| 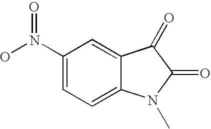 | UA19999 and/or NS199999 | μ17.1 | 33 μM |
| 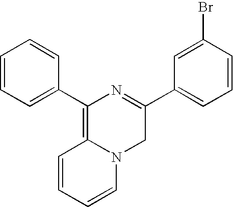 | UA21497 | −20.3 | 0.1 μM |
| 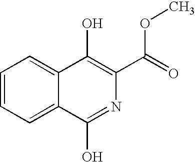 | UA45336 and/or NS45336 | −20.3 | 12.5 μM |
| 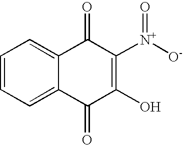 | UA45609 and/or NS45609 | −17.8 | 17 μM |

### Example 10

### Lipid Phosphatase Activity of PRL-1

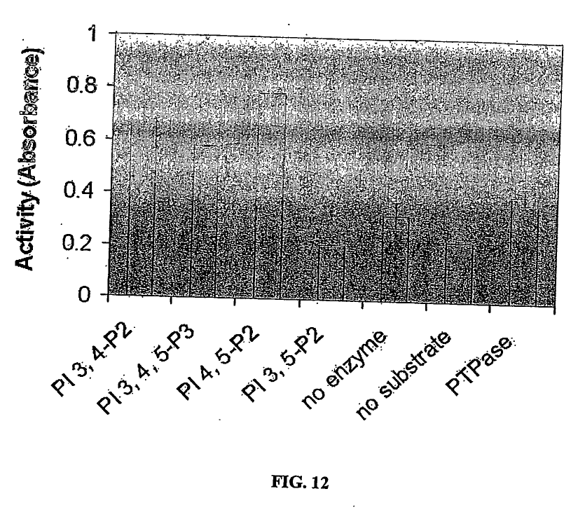

Since the molecular modeling study showed that PRL-1 shares a similar structure with the lipid phosphatase PTEN, PRL-1 was tested for possible lipid phosphatase activity. The results are rather intriguing. As shown in FIG. 12, PRL-1 exhibited very strong lipid phosphatase activity compared to its PTPase activity. Most interestingly, the lipid phosphatase activity of PRL-1 is specific to 4-phosphate. PRL-1 produced free phosphate when phosphatidylinositol 3,4,5-trisphosphate (PI 3,4,5-P₃), PI 3,4-P₂, and PI 4,5-P₂ were used as substrates but failed to do so when PI 3,5-P₂ Was used as substrate (FIG. 12). This activity is different from that of PTEN which is an inositol 3-phosphatase. It is well known that 3-phosphatases and 5-phosphatases are key players in the insulin signaling pathway. The significance of this 4-phosphatase activity of PRL-1 remains to be studied.

### Example 11

### Analogs for the Inhibition of PRL-1 Phosphatase

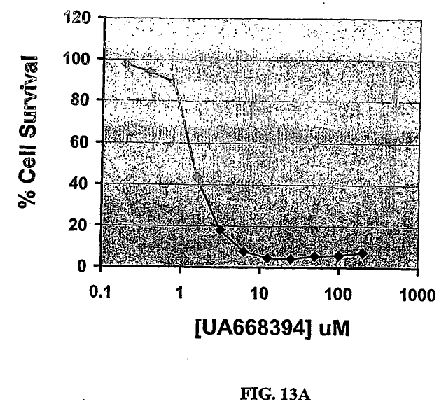

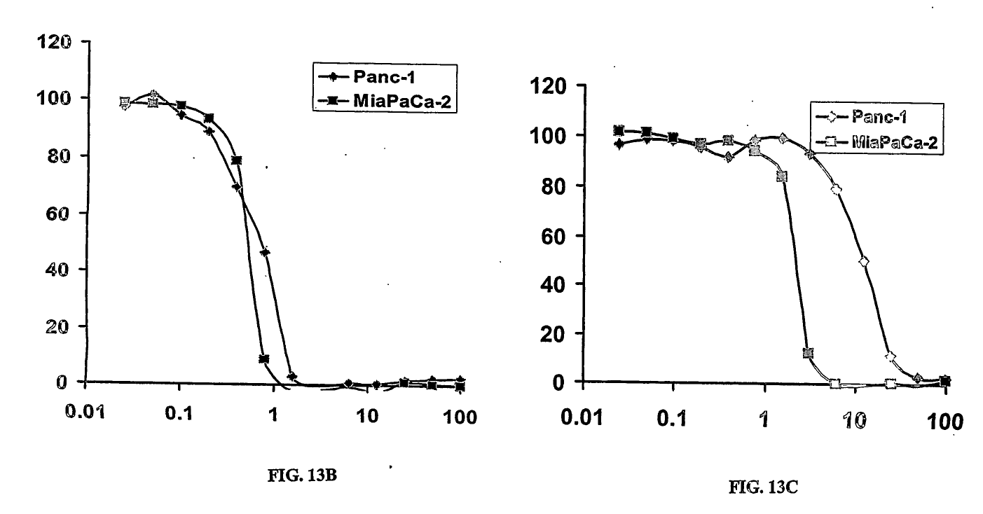

UA668394 was previously identified as a PRL-1 phosphatase inhibitor (IC₅₀=7 μM) using high throughout library screening. UA668394 was found to inhibit the growth of the pancreatic cancer cell line MiaPaCa-2 at an IC₅₀ of 1.2 μM, using a MTS cell proliferation assay (FIG. 13A). To obtain more potent inhibitors of PRL-1 with higher anti-pancreatic cancer activities, a series of analogues of UA668394 were used (Table 5).

Analogs of UA668394 were identified and synthesized Further studies were conducted to determine the ability of these compounds to inhibit cell proliferation in pancreatic cancer cells. Pancreatic cancer cells, Panc-1 and Mia PaCa-2, were treated with UA668394-1 and UA66839-2 analogs, as described above. The results showed that compared to UA668394, the UA668394-1 analog was a better PRL-1 inhibitor compound. Specifically, UA668394-1 (HT-8) has submicromolar IC₅₀ against MiaPaCa-2 and Panc-1 pancreatic cancer cells (0.5 μM and 0.7 μM, respectively)(FIG. 13B), while UA668394-2 (HT-11), an isomer of UA668394, showed very similar activity to that of UA668394 (IC₅₀=2.2 μM in MiaPaCa-2 cells)(FIG. 13C). The fluorine or chloride substituted compounds (see Table 5) are designed to lower the molecular weight, increase the bioavailability and lower the non-specific binding. All compounds are evaluated in a cell free PRL-1 assay for enzymatic inhibition.

### Example 12

### Purification of Recombinant PRL-1 Protein Using Ni-NTA Column

To produce active PRL-1 protein for in vitro PRL-1 phosphatase assays, Invitrogen's Ni-NTA Purification System (Invitrogen), was used to purify recombinant PRL-1 protein expressed in bacteria.

The inventors first cloned the full length open reading frame of PRL-1 to the bacteria expression vector pProEx-HTa (Invitrogen) under the control of an IPTG inducible promoter. A six-histidine tag was added to the C-terminus of PRL-1 for quick purification of PRL-1 using the Ni-NTA system. The expression vector was transformed to bacteria strain BL21 and checked for PRL-1 expression using Western blot. For large scale expression, bacteria were grown in 500 ml LB media to logarithm phase (OD₆₀₀ between 0.5 to 0.9) and induced to express PRL-1 by adding IPTG to final concentration of 1 mM and incubating for 4 hours. Bacteria were harvested by centrifugation at 5,000 rpm for 5 min and resuspended in the Native Binding Buffer (50 mM NaPO4, 0.5M NaCl, pH8.0, and 10 mM imidazole) at 16 ml/100 ml culture. The bacteria cells were then lysated by adding lmg/ml lysozyme and sonication. The cell lysate was centrifuged at 3,000 g for 15 min and the supernatant were then transferred to a 10-ml column pre-packed with 1.5 ml of Ni-NTA resin (Invitrogen). The column was gently agitated for 60 min to allow the binding of the His-tagged PRL-1 to the resin. After the binding reaction, the column was washed with the Native Wash Buffer (50 mM NaPO4, 0.5M NaCl, pH 8.0, and 20 mM imidazole) for 4 times. Finally, the PRL-1 protein was eluted off the column with 10 ml of Native Elution Buffer (50 mM NaPO4, 0.5 M NaCl, pH 8.0, and 250 mM imidazole). 0.5 ml fractions were collected and analysed by SDS-PAGE. The fractions containing the PRL-1 protein were combined and stored at 4° C. or −20° C. with the addition of 30% glycerol. The concentration of the protein was estimated by measuring the absorbance at OD₂₈₀ using a spectrophotometer. The activity of the protein was evaluated by the enzymatic PRL-1 assay.

### Example 13

### Inhibition of PRl-1 Expression by siRNA

siRNA oligonucleotides specific to the PRL-1 mRNA were used to suppress the expression of PRL-1 gene. To achieve maximal suppression, a mixture of 4 siRNA oligonucleotides that target different regions of the PRL-1 mRNA were used. These siRNA were designed and synthesized by the Dharmacon RNA Technologies (Lafayette, CO). Each of the siRNA oligonucleotide duplexes was denatured and annealed individually before being mixed together in equal moles to form a siRNA oligonucleotide pool (SMARTPool, Dharmacon RNA Technologies). A stock solution of 20 μM was prepared and stored at −20° C.

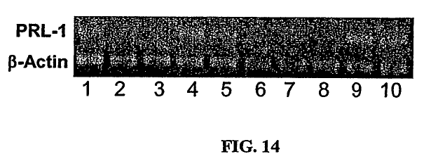

A transient transfection procedure was used to evaluate the inhibition of PRL-1 expression by the SMARTPool siRNA mixture. Briefly, MiaPaCa-2 cells were grown to 40-50% confluency the day of transfection in 6-well plates and washed with Dulbecco's phosphate bufferd saline (PBS buffer, Cellgro, Herdon, Va.). OPTI-MEM transfection media (Introgen, Carlsbad, Calif.) containing 3 μl of Lipofectin reagent (Invitrogen) per ml of media for each 100 nanomoles of siRNA oligonucleotides used was added to the cell culture plates. siRNA oligonucleotides were then added dropwise to obtain the final concentrations. Cells were incubated in transfection media for 6 hours, then washed once with PBS and given normal growth media. Cells were harvested with trypsinization. To evaluate the PRL-1 expression levels in the siRNA treated cells, total RNA was isolated from the harvested cells using SNAP RNA isolation kit (Invitrogen) and RT-PCR was carried out using the Omniscript RT kit (Qiagen, Valencia, Calif.). The β-actin transcript was also amplified in each reaction to serve as an internal control. As shown in FIG. 14, 72 hours past transfection the siRNA oligos suppressed expression of PRL-1 more than 90% at all three concentrations tested (50, 100 and 200 nM in lanes 6, 7 and 8 of FIG. 14).

### Example 14

### PTEN Assay

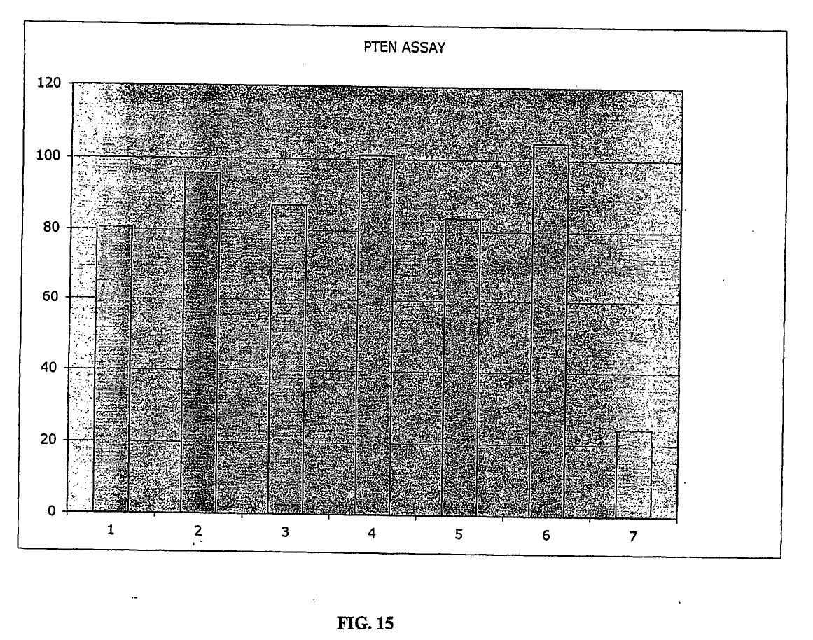

To further identify PRL-1 inhibitors the University of Arizona (UA) Natural Products Library was screened in a similar manner to that discussed above in Examples 8 and 9. Five additional PRL-1 inhibitors were identified (Table 6). Since the molecular modeling study showed that PRL-1 shares a similar structure with PTEN, as discussed in Example 10, these compounds were tested for PTEN activity. PTEN is an inositol 3-phosphatase which cleaves a phosphate from PI(3,4,5)P3. Studies were conducted to confirm that the compounds identified are specific for PRL-1 activity and not PTEN activity. Thus, a PTEN assay was conducted using malachite green as the substrate. Malachite green is known to form a complex with free phosphate. The plates were read at 630 nm using a spectrophotometer. As shown in FIG. 15, these compounds did not exhibit PTEN activity when compared to the DMSO control and sodium orthovandate NAVO₄ a positive control.

Next, the compounds identified were tested for their ability to inhibit PRL-1 activity. The UA64859, UA47548, UA63415 compounds exhibited 76%, 70% and 62% PRL-1 inhibitory activity, respectively. On the other hand, the least PRL-1 inhibition was observed with the UA61880 (50% inhibition) and UA58428 (42% inhibition) compounds.

TABLE 6

| STRUCTURE | NAME | INHIBITION % |
|---|---|---|
| 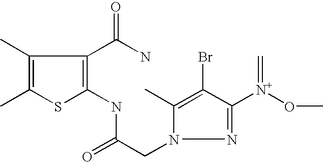 | UA47548 | 70% |
| 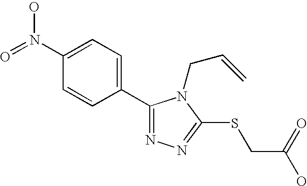 | UA58428 | 42% |
| 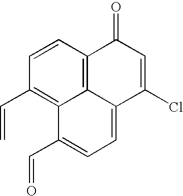 | UA61880 | 50% |
| 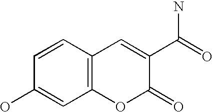 | UA63415 | 62% |
| 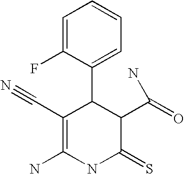 | UA64859 | 76% |

### Example 15

### Cell Proliferation by PRL-1 Inhibitors

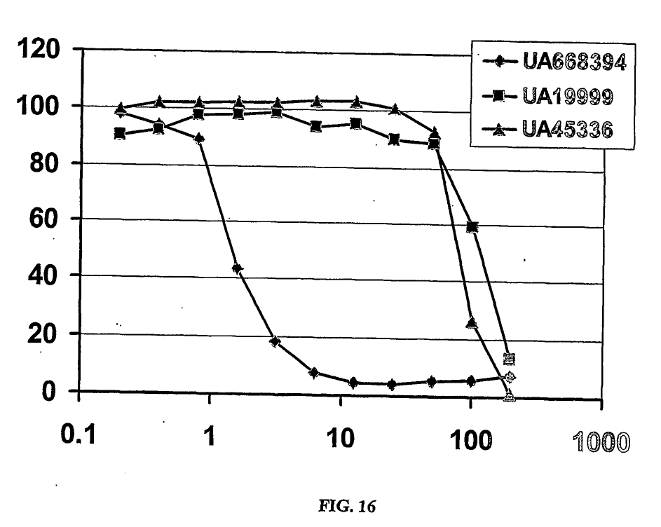

Compounds identified as being positive for anti-PRL-1 activity were tested for their ability to inhibit cell proliferation in human pancreatic cancer cells. The inhibitory activity of UA668394, UA19999 and UA45336 (Table 5) were examined. Cell proliferation assays were conducted using the pancreatic cancer cell line Mia PaCa-2, as described in Example 1. Briefly, 2.0-5.0×10⁵ cells were seeded in 100-mm culture dishes and allowed to attach overnight at 37° C. Adherent cells were washed and incubated with serum-free RPMI 1640 or RPMI containing 10% FBS for 48 h, and treated with the compound of interest to determine its effect on the inhibition of cell growth. For example, cells were treated with of the UA668394, UA19999 and UA45336 compounds and analyzed for inhibition of cell proliferative using a MTS assay. The data shows that the UA668394 compound was better at inhibiting cell proliferation than the UA19999 or UA45336 compounds. Specifically, the IC₅₀ of Mia PaCa-2 cells treated with the UA668394 compound was found to be 1.2 μM whereas, cells treated with the UA19999 and UA45336 compounds showed an IC₅₀ of 120 μM and 95 μM respectively (FIG. 16A). Thus, in Mia PaCa-2 pancreatic cancer cells the UA668394 compound was found to have the best overall inhibition of cell proliferation.

### Example 16

### MiaPaca Human Pancreatic In Vivo Xenograft Model

The antitumor effect of PRL-1 is assessed against the MiaPaca human pancreatic tumor model. MiaPaca tumors are implanted subcutaneously into the flanks of nude mice. As the tumors reach a predetermined size of approximately 100 mm³, the mice are randomized into therapy groups. UA668394-1 is administered by IV injection given for 5 daily doses at maximum tolerated dose (MTD), ½ MTD, ¼ MTD. Mean tumor volume are determined three times per week. Tumor volume is determined by caliper measurements (mm) and using the formula for an ellipsoid sphere: L×W²/2=mm³, where L is the length in mm and W is the width in mm. The formula is also used to calculate tumor weight (mg), assuming unit density (1 mm³=1 mg). The study is terminated when the tumor volumes in the control group(s) reach 2000 mm³. The time to reach evaluation size for the tumor of each animal is used to calculate the overall delay in the growth of the median tumor (T-C).

All of the compositions and/or methods and/or apparatus disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and/or apparatus and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,367,110
U.S. Pat. No. 4,415,732
U.S. Pat. No. 4,452,901
U.S. Pat. No. 4,458,066
U.S. Pat. No. 5,221,605
U.S. Pat. No. 5,238,808
U.S. Pat. No. 5,279,721
U.S. Pat. No. 5,310,687
U.S. Pat. No. 5,354,855
U.S. Pat. No. 5,795,715
U.S. Pat. No. 5,889,136
Bajorin et al., J. Clin. Oncol., 6(5):786-92, 1988.
Berzal-Herranz et al., Genes Dev., 6(1):129-134, 1992.
Bosher and Labouesse, Nat. Cell. Biol, 2:E31-E36, 2000.
Calaluce et al., Mol. Carcinog., 30:119-129, 2001.
Caplen et al., Gene, 252(1-2):95-105, 2000.
Cates et al., Cancer Lett., 110(1-2):49-55, 1996.
Cech et al., Cell, 27(3 Pt 2):487-496, 1981.
Chowrira et al., J. Biol. Chem., 268:19458-62, 1993.
Chowrira et al., J. Biol. Chem., 269(41):25856-25864, 1994.
Culver et al., Science, 256(5063):1550-1552, 1992.
De Jager et al., Semin. Nucl. Med., 23(2):165-179, 1993.
Diamond et al., Am. J. Physiol., 271(1-1):G121-9, 1996.
Diamond et al., Mol. Cell. Biol., 14(6):3752-3762, 1994.
Ditzel et al., Proc. Natl. Acad. Sci. USA, 94:8110-8115, 1997.
Doolittle and Ben-Zeev, Methods Mol Biol., 109:215-237, 1999.
Elbashir et al., Genes Dev., 5(2):188-200, 2001.
Elbashir et al., Nature, 411(6836):494-498, 2001.
Elchebly et al., Science, 283(5407):1544-1548, 1999.
Fire et al., Nature, 391:806-811, 1998.
Forster and Symons, Cell, 49(2):211-220, 1987.
Grishok et al., Science, 287:2494-2497, 2000.
Gulbis and Galand, Hum Pathol, 24(12):1271-85, 1993.
Harlow and Lane, Antibodies: A Laboratory manual, Cold Spring Harbor Laboratory, 1988.
Haseloff and Gerlach, Nature, 334:585-591, 1988.
Hu et al., Oncology, 64:160-165, 2003.
Joyce, Nature, 338:217-244, 1989.
Kerr et al., Br. J. Cancer, 26(4):239-257, 1972.
Ketting et al., Cell, 99:133-141, 1999.
Kim and Cech, Proc. Natl. Acad. Sci. USA, 84:8788-8792,1987.
Kononen et al., Nat. Med., 4(7):844-847,1998.
Krishan, J. Cell Biol., 66(1):188-93, 1975.
Lieber and Strauss, Mol. Cell. Biol., 15: 540-551, 1995.
Lin and Avery, Nature, 402:128-129, 1999.
Luttges et al., Histpathol., 32:444-448, 1998.
Martin et al, Exp. Hematol, 26:252-264, 1998.
Michel and Westhof, J. Mol. Biol., 216:585-610,1990.
Mitchell et al., Ann. NY Acad. Sci., 690:153-166, 1993.
Mitchell et al., J. Clin. Oncol., 8(5):856-869, 1990.
Montgomery et al., Proc. Natl. Acad. Sci. USA, 95:155-2-15507, 1998.
Nakamura et al., In: Handbook of Experimental Immunology (4^{th} Ed.), Weir et al., (eds). 1:27, Blackwell Scientific Publ., Oxford, 1987.
Palukaitis et al., Virology, 99:145-151, 1979.
Patani and LaVoie, Chem. Rev., 96:3147-3178, 1996.
PawsonTrends Genet., 17(11-12):343-5, 1994.
PCT Appl. WO 00/44914
PCT Appl. WO 01/36646
PCT Appl. WO 01/68836
PCT Appl. WO 84/03564
PCT Appl. WO 99/32619
Perriman et al., Gene, 113:157-163,1992.
Perrotta and Been, Biochemistry, 31(1):16-21, 1992.
Prody et al., Science, 231:1577-1580, 1986.
Rafie et al., Pancreas, 7:123-131, 1992.
Ravindranath and Morton, Intern. Rev. Immunol., 7: 303-329, 1991.
Reinhold-Hurek and Shub, Nature, 357:173-176, 1992.
Remington's Pharmaceutical Sciences, 18th Ed. Mack Printing Company, 1990.
Rozen and Skaletsky, Methods Mol. Biol., 132:365-386, 2000.
Sambrook et al., In: Molecular cloning, Cold Spring Harbor Laboratory Press, Cold Spring Harbor, 2001.
Sarver et al., Science, 247:1222-1225, 1990.
Scanlon et al., Proc. Natl. Acad. Sci. USA, 88:10591-10595, 1991.
Sharp and Zamore, Science, 287:2431-2433, 2000.
Sharp, Genes Dev., 13:139-141, 1999.
Sioud et al., J. Mol. Biol., 223:831-835, 1992.
Symons, Annu. Rev. Biochem., 61:641-671, 1992.
Tabara et al., Cell, 99:123-132, 1999.
Thompson et al., Nature Genet., 9:444-450, 1995.
Tonks et al., Cell, 87(3):365-368, 1996.
Wang et al., Cancer, 88:2787-2795, 2000.
Wang et al., J. Biol. Chem., 277(48):46659-46668, 2002.
Wincott et al., Nucleic Acids Res., 23(14):2677-2684, 1995.
Yuan and Altman, Science, 263:1269-1273, 1994.
Yuan et al., Proc. Natl. Acad. Sci. USA, 89:8006-8010, 1992.
Zeng et al., Biochem. Biophys Res. Commun., 244(2):421-427, 1998.

1. A method of diagnosing or predicting development of pancreatic cancer in a subject comprising:
(a) obtaining a cell-containing sample from said subject; and
(b) assessing PRL-1 activity or expression in a cell of said cell sample,
wherein increased activity or expression of PRL-1 in said cell, when compared to a normal cell of the same type, indicates that said subject has or is at risk of developing pancreatic cancer.

2. The method of claim 1, wherein said cell is a tumor cell.

3. The method of claim 1, wherein assessing comprises assessing PRL-1 expression.

4. The method of claim 3, wherein assessing PRL-1 expression comprises Northern blotting.

5. The method of claim 3, wherein assessing PRL-1 expression comprises quantitative RT-PCR.

6. The method of claim 3, wherein assessing PRL-1 expression comprises Western blotting.

7. The method of claim 3, wherein assessing PRL-1 expression comprises quantitative immunohistochemistry.

8. The method of claim 1, wherein assessing comprises assessing PRL-1 activity.

9. The method of claim 1, wherein said subject has previously been diagnosed with cancer.

10. The method of claim 1, wherein said subject has not previously been diagnosed with cancer and appears cancer free at the time of testing.

11. The method of claim 1, further comprising administering a prophylactic cancer treatment to said subject following testing.

12. The method of claim 1, further comprising administering a cancer therapy to said subject following testing.

13. The method of claim 12, wherein said cancer therapy is a chemotherapy, a radiotherapy, an immunotherapy, a gene therapy, a hormonal therapy or surgery.

14. A method of predicting the efficacy of a cancer therapy comprising:
(a) administering a cancer therapy to said subject;
(b) obtaining a tumor cell-containing sample from said subject; and
(c) assessing PRL-1 activity or expression in a tumor cell of said sample,
wherein decreased activity or expression of PRL-1 in said tumor cell, when compared to a tumor cell of the same type prior to treatment, indicates that said therapy is efficacious.

15. The method of claim 14, wherein assessing PRL-1 expression comprises measuring PRL-1 protein levels.

16. The method of claim 14, wherein assessing PRL-1 expression comprises measuring PRL-1 transcript levels.

17. The method of claim 14, further comprising assessing PRL-1 activity or expression at multiple time points.

18. A method of screening a candidate compound for anti-cancer activity comprising:
(a) providing a cell;
(b) contacting said cell with a candidate compound; and
(c) assessing the effect of said candidate compound on PRL-1 expression or activity,
wherein a decrease in the amount of PRL-1 expression or activity, as compared to the amount of PRL-1 expression or activity in a similar cell not treated with said candidate compound, indicates that said candidate compound has anti-cancer activity.

19. The method of claim 18, wherein said candidate compound is a protein, a nucleic acid or a organo-pharmaceutical.

20. The method of claim 18, wherein said cell is a tumor cell.

21. The method of claim 18, wherein assessing comprises assessing PRL-1 expression.

22. The method of claim 21, wherein assessing PRL-1 expression comprises Northern blotting.

23. The method of claim 21, wherein assessing PRL-1 expression comprises quantitative RT-PCR.

24. The method of claim 21, wherein assessing PRL-1 expression comprises Western blotting.

25. The method of claim 21, wherein assessing PRL-1 expression comprises quantitative immunohistochemistry.

26. The method of claim 18, wherein assessing comprises assessing PRL-1 activity.

27. A method of treating cancer comprising administering to a subject in need thereof a composition that inhibits PRL-1 activity.

28. The method of claim 27, wherein said compound inhibits PRL-1 expression.

29. The method of claim 27, wherein said candidate compound is a protein, a nucleic acid or an organo-pharmaceutical.

30. The method of claim 29, wherein said protein is an antibody that binds immunologically to PRL-1.

31. The method of claim 29, wherein said nucleic acid is a PRL-1 antisense nucleic acid, a PRL-1 RNAi nucleic acid, or an antibody encoding a single-chain antibody that binds immunologically to PRL-1.

32. The method of claim 27, wherein said cancer is selected from the group consisting of pancreatic cancer, leukemia, ovarian cancer, breast cancer, lung cancer, colon cancer, liver cancer, prostate cancer, testicular cancer, stomach cancer, brain cancer, bladder cancer, head & neck cancer, and melanoma.

33. The method of claim 27, further comprising administering a second cancer therapy to said subject.

34. The method of claim 33, wherein said second cancer therapy is a chemotherapy, a radiotherapy, an immunotherapy, a gene therapy, a hormonal therapy or surgery.

35. The method of claim 27, wherein said composition is administered more than once.

36. A method of diagnosing or predicting development of pancreatic cancer in a subject comprising subjecting said subject to whole body scanning for PRL-1 activity or expression in a cell.

37. A method of monitoring an anticancer therapy comprising assessing the expression or function of PRL-1 in a cancer cell of a subject following or during provision of said anticancer therapy.


SEQUENCE LISTING (19 sequences)

SEQ ID NO: 1 - RNA, 21 nt, Artificial Sequence
  Description of Artificial Sequence  Synthetic Primer
aaacaaauuuauagaggaacu

SEQ ID NO: 2 - DNA, 21 bp, Artificial Sequence
  Description of Combined DNA/RNA Molecule Isolated RNA comprising a nucleotide sequence
  Description of Artificial Sequence  Synthetic Primer
acaaauuuauagaggaacutt

SEQ ID NO: 3 - DNA, 21 bp, Artificial Sequence
  Description of Combined DNA/RNA Molecule Isolated RNA comprising a nucleotide sequence
  Description of Artificial Sequence  Synthetic Primer
aguuccucuauaaauuugutt

SEQ ID NO: 4 - RNA, 21 nt, Artificial Sequence
  Description of Artificial Sequence  Synthetic Primer
aacaaauuuauagaggaacuu

SEQ ID NO: 5 - DNA, 20 bp, Artificial Sequence
  Description of Combined DNA/RNA Molecule Isolated RNA comprising a nucleotide sequence
  Description of Artificial Sequence  Synthetic Primer
caauuuauagaggaacuutt

SEQ ID NO: 6 - DNA, 21 bp, Artificial Sequence
  Description of Combined DNA/RNA Molecule Isolated RNA comprising a nucleotide sequence
  Description of Artificial Sequence  Synthetic Primer
aaguuccucuauaaauuugtt

SEQ ID NO: 7 - RNA, 21 nt, Artificial Sequence
  Description of Artificial Sequence  Synthetic Primer
aaagaagguauccauguucuu

SEQ ID NO: 8 - DNA, 21 bp, Artificial Sequence
  Description of Combined DNA/RNA Molecule Isolated RNA comprising a nucleotide sequence
  Description of Artificial Sequence  Synthetic Primer
agaagguauccauguucuutt

SEQ ID NO: 9 - DNA, 21 bp, Artificial Sequence
  Description of Combined DNA/RNA Molecule Isolated RNA comprising a nucleotide sequence
  Description of Artificial Sequence  Synthetic Primer
aagaacauggauaccuucutt

SEQ ID NO: 10 - RNA, 21 nt, Artificial Sequence
  Description of Artificial Sequence  Synthetic Primer
aaauacgaagaugcaguacaa

SEQ ID NO: 11 - DNA, 21 bp, Artificial Sequence
  Description of Combined DNA/RNA Molecule Isolated RNA comprising a nucleotide sequence
  Description of Artificial Sequence  Synthetic Primer
auacgaagaugcaguacaatt

SEQ ID NO: 12 - DNA, 21 bp, Artificial Sequence
  Description of Combined DNA/RNA Molecule Isolated RNA comprising a nucleotide sequence
  Description of Artificial Sequence  Synthetic Primer
uuguacugcaucuucguautt

SEQ ID NO: 13 - RNA, 21 nt, Artificial Sequence
  Description of Artificial Sequence  Synthetic Primer
aagaugcaguacaauucauaa

SEQ ID NO: 14 - DNA, 21 bp, Artificial Sequence
  Description of Combined DNA/RNA Molecule Isolated RNA comprising a nucleotide sequence
  Description of Artificial Sequence  Synthetic Primer
gaugcaguacaauucauaatt

SEQ ID NO: 15 - DNA, 21 bp, Artificial Sequence
  Description of Combined DNA/RNA Molecule Isolated RNA comprising a nucleotide sequence
  Description of Artificial Sequence  Synthetic Primer
uuaugaauuguacugcauctt

SEQ ID NO: 16 - RNA, 21 nt, Artificial Sequence
  Description of Artificial Sequence  Synthetic Primer
aauucauaagacaaaagcggc

SEQ ID NO: 17 - DNA, 21 bp, Artificial Sequence
  Description of Combined DNA/RNA Molecule Isolated RNA comprising a nucleotide sequence
  Description of Artificial Sequence  Synthetic Primer
uucauaagacaaaagcggctt

SEQ ID NO: 18 - DNA, 21 bp, Artificial Sequence
  Description of Combined DNA/RNA Molecule Isolated RNA comprising a nucleotide sequence
  Description of Artificial Sequence  Synthetic Primer
gccgcuuuugucuuaugaatt

SEQ ID NO: 19 - DNA, 21 bp, Artificial Sequence
  Description of Combined DNA/RNA Molecule Isolated RNA comprising a nucleotide sequence
  Description of Artificial Sequence  Synthetic Primer
ttuguuuaaauaucuccuuga